SEQUENCE LISTING (25 sequences)

SEQ ID NO: 1 - protein, 102 aa, Theobroma cacao
YGRKQYERDPRQQYEQCQRRCESEATEEREQEQCEQRCEREYKEQQRQQEEELQRQYQQCQGRCQEQQQGQREQQQCQRKCWEQYKEQERGEHENYHNHKKN

SEQ ID NO: 2 - protein, 100 aa, synthetic construct
  REGION 1..100: note = peptide according to an embodiment of the invention
YGRKQYERDPRQQYEQCQRRCESEATEEREQEQCEQRCEREYKEQQRQQEEELQRQYQQCQGRCQEQQQGQREQQQCQRKCWEQYKEQERGEHENYHNHK

SEQ ID NO: 3 - protein, 28 aa, synthetic construct
  REGION 1..28: note = signal peptide according to an embodiment of the invention
MVISKSPFIVLIFSLLLSFALLCSGVSA

SEQ ID NO: 4 - protein, 130 aa, synthetic construct
  REGION 1..130: note = (peptide according to an embodiment of the invention
MVISKSPFIVLIFSLLLSFALLCSGVSAYGRKQYERDPRQQYEQCQRRCESEATEEREQEQCEQRCEREYKEQQRQQEEELQRQYQQCQGRCQEQQQGQREQQQCQRKCWEQYKEQERGEHENYHNHKKN

SEQ ID NO: 5 - protein, 128 aa, synthetic construct
  REGION 1..128: note = peptide according to an embodiment of the invention
MVISKSPFIVLIFSLLLSFALLCSGVSAYGRKQYERDPRQQYEQCQRRCESEATEEREQEQCEQRCEREYKEQQRQQEEELQRQYQQCQGRCQEQQQGQREQQQCQRKCWEQYKEQERGEHENYHNHK

SEQ ID NO: 6 - protein, 47 aa, Theobroma cacao
YERDPRQQYEQCQRRCESEATEEREQEQCEQRCEREYKEQQRQQEEE

SEQ ID NO: 7 - protein, 60 aa, Theobroma cacao
LQRQYQQCQGRCQEQQQGQREQQQCQRKCWEQYKEQERGEHENYHNHKKNRSEEEEGQQR

SEQ ID NO: 8 - DNA, 27 bp, synthetic construct
  misc_feature 1..27: note = Forward_BamH1 forward primer
  source 1..27: mol_type = other DNA
cgcggatcctatggcagaaaacaatat

SEQ ID NO: 9 - DNA, 27 bp, synthetic construct
  misc_feature 1..27: note = Reverse_Kpn1 forward primer
  source 1..27: mol_type = other DNA
cgcggtacctttgtgattatggtaatt

SEQ ID NO: 10 - protein, 6 aa, synthetic construct
  REGION 1..6: note = HQ tag
HQHQHQ

SEQ ID NO: 11 - protein, 12 aa, synthetic construct
  REGION 1..12: note = HN tag
HNHNHNHNHNHN

SEQ ID NO: 12 - protein, 19 aa, synthetic construct
  REGION 1..19: note = HAT tag
KDHLIHNVHKEEHAHAHNK

SEQ ID NO: 13 - protein, 13 aa, synthetic construct
  REGION 1..13: note = ALFA tag
SRLEEELRRRLTE

SEQ ID NO: 14 - protein, 15 aa, synthetic construct
  REGION 1..15: note = Avi tag
GLNDIFEAQKIEWHE

SEQ ID NO: 15 - protein, 4 aa, synthetic construct
  REGION 1..4: note = C-tag
EPEA

SEQ ID NO: 16 - protein, 26 aa, synthetic construct
  REGION 1..26: note = Calmodulin-tag
KRRWKKNFIAVSAANRFKKISSSGAL

SEQ ID NO: 17 - protein, 13 aa, synthetic construct
  REGION 1..13: note = E-tag
GAPVPYPDPLEPR

SEQ ID NO: 18 - protein, 8 aa, synthetic construct
  REGION 1..8: note = FLAG-tag
DYKDDDDK

SEQ ID NO: 19 - protein, 9 aa, synthetic construct
  REGION 1..9: note = HA-tag
YPYDVPDYA

SEQ ID NO: 20 - protein, 10 aa, synthetic construct
  REGION 1..10: note = Myc-tag
EQKLISEEDL

SEQ ID NO: 21 - protein, 18 aa, synthetic construct
  REGION 1..18: note = NE-tag
TKENPRSNQEESYDDNES

SEQ ID NO: 22 - protein, 9 aa, synthetic construct
  REGION 1..9: note = Rho1D4 tag
TETSQVAPA

SEQ ID NO: 23 - protein, 15 aa, synthetic construct
  REGION 1..15: note = S-tag
KETAAAKFERQHMDS

SEQ ID NO: 24 - protein, 37 aa, synthetic construct
  REGION 1..37: note = SBP tag
DEKTTGWRGGHVVEGLAGELEQLRARLEHHPQGQREP

SEQ ID NO: 25 - protein, 8 aa, synthetic construct
  REGION 1..8: note = Strep-tag
WSHPQFEK

(19) United States
(12) Patent Application Publication
(10) Pub. No.: US 2025/0296963 A1
(43) Pub. Date: Sep. 25, 2025

(54) PEPTIDE HAVING ANTIMICROBIAL ACTIVITY

(71) Applicant: CABOSSE NATURALS NV, Halle (BE)

(72) Inventors: Gabi KOPP, Halle (BE); Herwig BERNAERT, Halle (BE); Matthias ULLRICH, Bremen (DE); Nikolai KUHNERT, Bremen (DE); Neha KUMARI, Radeberg (DE)

(73) Assignee: CABOSSE NATURALS NV, Halle (BE)

(21) Appl. No.: 18/863,463

(22) PCT Filed: May 10, 2023
(86) PCT No.: PCT/EP2023/062397

§ 371 Date: Nov. 6, 2024

(30) Foreign Application Priority Data
May 10, 2022 (EP) 22172622.7

(51) Int. Cl. C07K 14/415; A61K 38/00; A61P 31/04; A61P 31/10; C12P 21/02

(52) U.S. Cl. CPC C07K 14/415; A61P 31/04; A61P 31/10; C12P 21/02; A61K 38/00

(57) ABSTRACT

The present invention is directed to a peptide, wherein said peptide has an amino acid sequence exhibiting at least 95% sequence identity to SEQ ID N° 1 or SEQ ID N° 2. The current invention also discloses compositions comprising said peptides, as well as specific uses.

### FIELD OF THE INVENTION

The invention pertains to the technical field of antimicrobial peptides, more particularly natural plant-derived peptides, which exhibit antimicrobial activity against various bacterial and fungal strains. In particular, the present invention relates to a natural, recombinant, or synthetic peptide derived from the cocoa plant vicilin protein. The invention further relates to the use of the peptide for the treatment of bacterial and fungal infections in plants, animals, and humans.

### BACKGROUND

Bacterial antibiotic resistance is a significant issue faced by the food and agricultural industries, the medical and veterinary professions, and others. The potential for transfer of potentially lethal antibiotic-resistant bacteria from a food animal to a human consumer is of particular concern.

Current methods for controlling the development and spread of antibiotic-resistant bacteria include changes in antibiotic usage and patterns of usage of different antibiotics, increased government surveillance and regulation, and continued development of new and improved antibiotics. However, the ability of most bacteria to adapt to antibiotic usage and to acquire resistance to existing and new antibiotics often overcomes such conventional measures and requires the continued development of alternative means for control of antibiotic resistance in bacteria.

Antimicrobial peptides (AMPs) are a diverse group of natural compounds present in animals, plants, insects, and microorganisms. These peptides are responsible for a defense against (other) microorganisms and, as such, they may be further utilized as an alternative to the chemical preservatives. However, their use in the treatment of plant, human, and/or animal microbial infestations is up to date limited for poor availability in nature and high cost of production, low stability, and complex formulation into the suitable therapeutic product. Thus, the use of antimicrobial peptides in crop protection as well as for therapeutic applications in human and veterinary medicine is still limited.

Antimicrobial proteins exhibit a variety of three-dimensional structures, which will determine in large part their activity and stability. The stability of a certain protein is typically a critical parameter for clinical and/or industrial applications.

The initial interest in plant-derived molecules which are AMPs was followed by isolation of purothionin, the first plant-derived AMP. Caleya et al., in Appl. Microbiol, 1972, 23(5) 998-100, reported purothionin, the low molecular weight protein from wheat and barley flour, as an AMP effective against a number of phytopathogenic bacteria such as Pseudomonas solanacearum, Xanthomonas phaseoli, X. campestris, Erwinia amylovora, and five Corynebacterium strains. Since then, several major groups of AMPs including thionins (types I-V), defensins, cyclotides, 2S albumin-like proteins, and lipid transfer proteins were discovered.

Plant vicilins are a commonly occurring class of plant seed storage protein. It has been shown that some vicilins are processed to produce plant defense peptides. The best characterized antimicrobial peptides produced from a vicilin are those of Macadamia integrifolia. Macadamia nut kernels contain a 666 aa vicilin protein that includes a 212 aa highly hydrophilic region proximal to an N-terminal signal sequence.

WO1998027805 discloses a family of vicilin type peptides with antimicrobial properties. Prototype proteins are of a natural origin and isolated from Macadamia integrifolia, as well as from some other species, including Theobroma cacao. In particular, two sequences marked as the 47-amino acid TcAMP1 (Theobroma cacao antimicrobial protein 1) and the 60-amino acid TcAMP2 (Theobroma cacao antimicrobial protein 2) were derived from a cocoa vicilin seed storage protein gene sequence which encodes 566 amino acids (aa), and recombinantly expressed in Escherichia coli. However, the isolated recombinant peptides and the compositions prepared thereof had effective antimicrobial properties at high doses (5-20 μg/ml).

Marcus et al., Plant. Mol. Biol Rep (2008) 26, 75-87 investigated three Macadamia integrifolia and two Theobroma cacao peptide sequences identified in the N-proximal hydrophilic region of vicilin seed protein. The peptide's antimicrobial activity was predicted based on the presence of the characteristic C-X-X-X-C-(10-12)-C-X-X-X-C motifs in the hydrophilic regions proximal to the N-terminus. His-tagged versions of the putative peptides were expressed in Escherichia coli. The obtained recombinant peptides were shown to have in vitro antimicrobial activity against six plant pathogen strains but the effective dose was very high.

Ecuador is the most important producer of fine flavored cocoa, accounting for roughly 50% of the world production. The fine flavored cocoa type produced in Ecuador is predominantly belonging to the Nacional variety (sometimes also called National or Arriba). Besides the intensively aromatic and traditional Nacional cocoa, a cocoa clone, CCN-51 has been cultivated in Ecuador since the 1960s. Unlike Nacional, CCN-51 is considered a bulk cocoa type because of its weaker aroma. However, it has a higher tolerance to changing climatic conditions, is resistant to different pathogens, and gives higher yields than other cocoa types. In consequence, the CCN-51 hybrid is very popular for cultivation by Ecuadorian farmers. US 2004/0172683 describes several polypeptides from the seed of cocoa bean responsible for the cocoa flavor.

A Vicilin-derived peptide that is used for the production of cocoa and chocolate as a cocoa flavor enhancer is known from WO2002086125A2.

The present invention describes thus an unknown peptide derived from cocoa that is highly effective and active against a large spectrum of animal and plant pathogenic microorganisms. The peptide is safe to use, preferably without containing aggressive chemicals, which can be toxic and environmentally harmful.

### SUMMARY OF THE INVENTION

The present invention and embodiments thereof serve to provide a solution to one or more of the above-mentioned disadvantages. To this end, the present invention relates to a peptide having antimicrobial activity according to claim 1. More specifically, the present invention provides a peptide exhibiting at least 95% sequence identity to SEQ ID N° 1 or SEQ ID N° 2. It has been surprisingly found that this peptide, obtainable from Theobroma cacao or being recombinantly or synthetically produced has antibacterial and/or antifungal activity against a broad spectrum of pathogens. Preferred embodiments of the peptide are shown in any of the claims 2 to 9.

In a second aspect, the present invention relates to a composition comprising a peptide according to claim 10. Preferred embodiments of the composition are shown in claims 11 and 12.

In a third aspect, the present invention relates to the use of the peptide for therapeutic purposes according to claim 13. Preferred embodiments of the use are shown in any of the claims 14 to 21. More particular, the peptide is used for infections with Gram-positive bacteria, Gram-negative bacteria, and/or fungi, preferably hyphal fungi or yeasts in humans, animals, or plants.

In a fourth aspect, the present invention relates to a method of obtaining said peptide, according to claims 22 and 23.

In a fifth aspect, the present invention relates to a vector comprising the genetic sequence encoding the peptide, that allows the expression of said peptide, according to claim 24, to a transgenic plant according to claim 25 and a method of obtaining the transgenic plant according to claim 26.

In a final aspect, the present invention relates to a seed coated with said peptide, according to claim 27.

### DESCRIPTION OF FIGURES

FIG. 1 shows proteins isolated from cocoa beans after separation on a two-dimensional (2D) SDS polyacrylamide gel. Horizontal separation is according to their isoelectric point (pI, pH 4-7) and vertical separation is based on their molecular weight. The highlighted spot was analyzed by peptide mass fingerprinting as revealed in FIG. 2A. The numbers on the left indicate the molecular weight in kDa of the reference marker.

FIG. 2 shows a representative scheme for the detection and sequence location of peptide mass fingerprinting fragments as determined by MALDI-TOF analysis. Underlined amino acid sequences in FIG. 2A were identified as tryptic digestion products of a peptide according to SEQ ID N° 1 obtained from a 2D SDS polyacrylamide gel of cocoa proteins. Underlined amino acid sequences in FIG. 2B were identified as tryptic digestion products of a recombinant peptide according to SEQ ID N° 2 obtained from an SDS polyacrylamide gel.

FIG. 3 shows the LC-MS results of a sample containing a peptide according to SEQ ID N° 2. The peptide detected had a mass of 15.3 kDa in both the non-reduced (FIG. 3A) and reduced (FIG. 3B) samples.

FIG. 4 shows the cytotoxicity of a peptide according to SEQ ID N° 2 for Candida albicans and HaCaT cells according to a lactate dehydrogenase (LDH) release assay. Each experiment was conducted with 10,000 cells that were incubated with the peptide at different concentrations for 1 h.

### DETAILED DESCRIPTION OF THE INVENTION

The present invention concerns a peptide or composition comprising said peptide as an active ingredient for medical or pharmaceutical use, wherein said peptide has an amino acid sequence according to SEQ ID N° 1 or SEQ ID N° 2. Furthermore, the present invention relates to the use of said peptide, to a method of obtaining said peptide, and to a vector that allows the expression of said peptide.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

The term “peptide,” as used herein, refers to any compound containing two or more amino acid residues joined by an amide bond formed from the carboxyl group of one amino acid residue and the amino group of the adjacent amino acid residue. The amino acid residues may have the L-form as well as the D-form, and may be naturally occurring or synthetic, linear as well as cyclic.

The term “therapeutically effective amount” is an amount that is effective to ameliorate a symptom of a disease. A therapeutically effective amount can be a “prophylactically effective amount” as prophylaxis can be considered therapy.

The cocoa bean “slurry” as used herein is defined as the grinded cocoa nibs mass after the separation of cocoa butter.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention and form different embodiments, as would be understood by those in the art. For example, in the following claims, any of the claimed embodiments can be used in any combination.

In a first aspect, the present invention relates to a peptide, wherein said peptide has an amino acid sequence exhibiting at least 80% sequence identity to SEQ ID N° 1 or SEQ ID N° 2. Preferably, said peptide has an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, 99%, 99.5% or more preferably 100% sequence identity to SEQ ID N° 1 or SEQ ID N° 2. In a preferred embodiment, the peptide has an amino acid sequence exhibiting at least 95% identity to SEQ ID N° 1 or SEQ ID N° 2.

The term “sequence identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity. A gap, i.e., a position in an alignment where a residue is present in one sequence but not in the other is regarded as a position with non-identical residues. Determining the percentage of sequence identity can be done manually, or by making use of computer programs that are available in the art. An example of a useful algorithm is PILEUP. Software for performing BLAST analyses is publicly available through the National Center for Biotechnology Information (http://www.ncbi.nlm.nih.gov/).

In another embodiment, said peptide has a sequence that differs with maximally 24, more preferably maximally 23, even more preferably maximally 22, even more preferably maximally 21, even more preferably maximally 20, even more preferably maximally 19, even more preferably maximally 18, even more preferably maximally 17, even more preferably maximally 16, even more preferably maximally 15, even more preferably maximally 14, even more preferably maximally 13, even more preferably maximally 12, even more preferably maximally 11, even more preferably maximally 10, even more preferably maximally 9, even more preferably maximally 8, even more preferably maximally 7, even more preferably maximally 6, even more preferably maximally 5, even more preferably maximally 4, even more preferably maximally 3, even more preferably maximally 2, even more preferably maximally 1 amino acid residues from one of the sequences SEQ ID N° 1 or SEQ ID N° 2.

In an embodiment, said peptide has an amino acid sequence according to SEQ ID N° 1 or that exhibits at least 98% sequence identity to SEQ ID N° 2 or that differs with maximally 2 amino acids from the sequence according to SEQ ID N° 2.

Alternatively, the peptide of the invention has a sequence identity according to SEQ ID N° 1 or N° 2.

Amino acid sequence variants of a peptide contemplated herein may be substitutional, insertional, or deletion variants. Deletion variants lack one or more residues of the peptide which may not be critical for function. Substitutional variants typically contain an alternative amino acid at one or more sites within the peptide and may be designed to modulate one or more properties of the polypeptide such as stability against proteolytic cleavage. Substitutions preferably are conservative, that is, one amino acid is replaced with one of similar size and side-chain or functional group. Conservative substitutions are well known in the art and include, for example, the changes of alanine to glycine, valine, or leucine; arginine to lysine; asparagine to glutamine; aspartate to glutamate; cysteine to methionine; glutamine to asparagine; glutamate to aspartate; glycine to proline; histidine to glutamine, tyrosine, arginine, lysine, asparagine or cysteine; isoleucine to leucine or valine; leucine to valine or isoleucine; lysine to arginine; phenylalanine to tyrosine, leucine or methionine; serine to threonine; threonine to serine; tryptophan to phenylalanine; tyrosine to tryptophan or phenylalanine; and valine to isoleucine or leucine.

By preference, the peptide of the invention is derived from or isolated from Theobroma cacao. More in particular, said peptide of the invention is derived from the N-terminal region of the vicilin protein of Theobroma cacao. Vicilin is a storage protein present and characterized in several plant species such as, but not limited to Ananas comosus, Arachis hypogaea, Beta vulgaris, Capsicum annuum, Capsicum chinense, Carya illinoinensis, Chenopodium quinoa, Corchorus olitorius, Cucurbita maxima, Fragaria vesca, Glycine max, Gossipum arboreum, Herrania umbratica, Hordeum vulgare, Jatropha curcas, Juglans regia, Macadamia integrifolia, Macleaya cordata, Musa acuminata, Papaver somniferum, Ricinus communis, Solanum lycopersicum, Spinacia oleracea, Stenocarpus sinuatus, Theobroma cacao and Zea mays. Such proteins are typically containing an extremely hydrophilic N-proximal region, and a C-terminal region. Additionally, they are produced carrying a hydrophobic N-terminal signal peptide, which is usually removed during protein maturation. The N-proximal region of said precursor protein is particularly interesting as it contains at least two, preferably four pairs of cysteine motifs (CXXXC) with the same spacing pattern.

In a further embodiment, the peptide as disclosed herein is derived from Theobroma cacao variety CCN-51. Surprisingly, the cacao variety CCN-51, known as highly resistant towards microbial infestations, was shown to be particularly rich in vicilin-derived N-terminal peptide.

In another or further embodiment, said peptide comprises a signal peptide at its N terminus. Preferably, said signal peptide has a sequence according to SEQ ID N° 3. In another embodiment, said signal peptide exhibits at least 95%, 96%, 97%, 98%, 99% or more preferably 100% sequence identity to SEQ ID N° 3. Alternatively, said peptide has a signal peptide sequence that differs with maximally 3, more preferably maximally 2, even more preferably 1 amino acid residues from the sequence SEQ ID N° 3. Vicilin proteins isolated from natural sources do not typically have said signal peptide. It has been surprisingly found that when a signal peptide was added to the peptide, its antimicrobial activity was enhanced.

In another embodiment, the peptide is a recombinant or a synthetic peptide. A suitable expression system, such as Escherichia coli, or another suitable system known in the art, upon insertion of a vector containing a coding sequence for a peptide with an SEQ ID N°1 or SEQ ID N°2, gives rise to a peptide. Any suitable protein/peptide synthesis method known in the art can be employed to synthesize the peptide of the invention.

In a further or another embodiment, the peptide as described herein is provided with an affinity tag at its N and/or C terminus. Affinity tags are used for affinity purification of recombinant proteins and peptides expressed in Escherichia coli and other systems. Said tag can be a poly-histidine-tag, which is an amino acid motif that consists of at least six histidine (His) residues.

In an embodiment, a peptide as disclosed herein containing a signal peptide has a sequence identity according to SEQ ID N°4 or N°5.

Alternatively and without being limitative, the peptide can be tagged with an HQ tag that alternates histidine and glutamine residues(HQHQHQ), an HN tag that alternates histidine and asparagine (HNHNHNHNHNHN), a HAT tag (KDHLIHNVHKEEHAHAHNK), an ALFA tag (SRLEEELRRRLTE), an Avi tag (GLNDIFEAQKIEWHE), a C-tag (EPEA), a Calmodulin-tag (KRRWKKNFIAVSAANRFKKISSSGAL), a polyglutamate tag, a polyarginine tag, an E-tag (GAPVPYPDPLEPR), a FLAG-tag (DYKDDDDK), an HA-tag (YPYDVPDYA), a Myc-tag (EQKLISEEDL), a NE-tag (TKENPRSNQEESYDDNES), a Rho1D4 tag (TETSQVAPA), an S-tag (KETAAAKFERQHMDS), an SBP tag (DEKTTGWRGGHVVEGLAGELEQLRARLEHHPQGQREP) or a Strep-tag (WSHPQFEK).

Analysis of the peptide of the invention showed that the peptide has antibacterial and/or antifungal activity.

The antimicrobial peptide per se has a particular three-dimensional structure which may be determined using X-ray crystallography or nuclear magnetic resonance techniques. Without wanting to be bound to theory, it is believed that this structure plays a role in the observed activity of said peptide. Alpha-helical plant-derived antimicrobial peptides (AMPs), to which the peptide of the invention belongs, often have amphipathic helices with one face of the helix predominantly hydrophilic and the other face predominantly hydrophobic. This structure is regularly found in AMPS that disrupt cell membranes to cause leakage of the cell content and lysis and in AMPs that enter the cells to attack other structures within the cell. Alpha-helical AMPs are usually structurally disordered in solution, which facilitates passage of the dense net of the cell wall. Upon binding to the cell membrane that is hydrophobic and charged below the lipid heads, secondary structures (alpha-helices) are formed which enables the protein to penetrate the cell membrane. This order/disorder transition is predominantly regulated by the length of the hydrophobic domain within the alpha-helices. Too long hydrophobic helices lead to ordered structures in solution which has a negative impact on AMP activity.

In an embodiment, said peptide is active against Gram-positive and Gram-negative bacteria and fungi, preferably hyphal fungi or yeasts. Without being limitative, the peptide is active against bacteria selected from the group comprising: Acinetobacter spp., Bartonella spp., Bordetella spp., Borrelia spp., Brucella spp., Campylobacter spp., Chlamydia spp., Clostridium spp., Corynebacterium spp., Enterococcus spp., Enterobacter spp., Erwinia spp., Escherichia spp., Francisella spp., Haemophilus spp., Helicobacter spp., Klebsiella spp., Legionella spp., Leptospira spp., Listeria spp., Mycobacterium spp., Mycoplasma spp., Neisseria spp., Rickettsia spp., Salmonella spp., Shigella spp., Staphylococcus spp., Streptococcus spp., Treponema spp., Ureaplasma spp., Vibrio spp., Yersinia spp., Acidovorax spp., Agrobacterium spp., Arthrobacter spp., Bacillus spp., Burkholderia spp., Clavibacter spp., Cronobacter spp., Curtobacterium spp., Lefisonia spp., Pantoea spp., Paenibacillus spp., Pectobacterium spp., Phytoplasma spp., Proteus spp., Pseudomonas spp., Ralstonia spp., Rhizobacter spp., Rhizomonas spp., Rhodococcus spp., Serratia spp., Sphingomonas spp., Spiroplasma spp., Streptomyces spp., Xanthomonas spp., Xylella spp., or Xylophilus spp.

Without wishing to be limitative, the peptide of the invention is active against fungi selected from the group comprising: Ajellomyces spp., Aspergillus spp., Basidiobolus spp., Blastomyces spp., Candida spp., Coccidioides spp., Conidiobolus spp., Cryptococcus spp., Emmonsia spp., Histoplasma spp., Hanseniaspora spp., Lacazia spp., Paracoccidioides spp., Pneumocystis spp., Sporothrix spp., Stachybotrys spp., Talaromyces spp., Acrocalymma spp., Aecidium spp., Albonectria spp., Allodus spp., Alternaria spp., Amphobotrys spp., Apiosporina spp., Armillaria spp., Blumeria spp., Botryotinia spp., Botrytis spp., Ceratosystis spp., Colletotrichum spp., Cryptosporiopsis spp., Exobasidium spp., Fusarium spp., Hypocrea spp., Leptosphaeria spp., Magnaporthe spp., Melampsora spp., Meyerozyma spp., Monilinia spp., Mycospharella spp., Microsphaera spp., Mucor ssp., Penicillium spp., Pichia spp., Phytophtora spp., Saccharomyces spp., Sporobolomyces spp., Plasmodiophora spp., Podosphaera spp., Puccinia spp., Pythium spp., Rhizoctonia spp., Sclerotinia spp., Septoria spp., Taphrina spp., Thanatephorus spp., Torulaspora spp., Uromyces spp., Ustilago spp., Venturia spp., or Verticillium spp.

In a second aspect, the present invention provides compositions comprising a peptide as described in the paragraphs above. This composition is particularly suitable for pharmaceutical and veterinary applications, for crop protection, and/or for cosmetical applications.

In an embodiment, said composition comprises a peptide exhibiting at least 95% sequence identity to SEQ ID N° 1 or SEQ ID N° 2 or that differs with maximally 5 amino acids from the sequence according to SEQ ID N° 1 or SEQ ID N° 2 and an excipient.

In some embodiments of the composition as disclosed herein, said peptide comprises a signal peptide having a sequence identity of at least 95% to SEQ ID N° 3 or has a sequence according to SEQ ID N° 3.

In an embodiment, the compositions are liquid, semisolid, solid, or gaseous and/or is in a dosage form of a tablet, capsule, powder, granulate, aerosol, paste, syrup, suspension, emulsion, or solution. Non-limiting examples of said compositions are soluble powders, soluble granules, wettable granules, tablet formulations, dry flowables, aqueous flowables, wettable dispersible granules, oil dispersions, suspension concentrates, dispersible concentrates, emulsifiable concentrates, aqueous suspensions, fertilizer granule, or sprayable compositions. Said compositions may be formulated such that it is suited for oral, injectable, intravenous, intramuscular, cutaneous, inhalation, topical or intranasal administration. In other embodiment, said compositions are formulated to allow coating of one or more objects, spraying, spray coating, evaporating, nebulising, atomising, suspending, diluting, etc.

In some embodiments, the compositions comprise a pharmaceutically acceptable carrier, excipient, or diluent. By preference, said excipients are chosen from fillers, binders, disintegrants, sweeteners, coatings, lubricants, and/or glidants. Diluents or fillers increase the bulk of solid compositions and may make a dosage form containing the compositions easier for the patient and caregiver to handle. Diluents suitable for tablets according to the current invention include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid compositions that are compacted into a dosage form, such as a tablet, may include excipients whose functions include helping to contain the active ingredient and other excipients together after compression. Suitable binders include acacia, alginic acid, carbomer (e.g. carbopol), carboxymethylcellulose sodium, dextrin, ethylcellulose, gelatin, guar gum, hydrogenated vegetable oil, hydroxyethyl cellulose, hydroxypropyl cellulose (e.g. Klucel®), hydroxypropyl methylcellulose (e.g. Methocel®), liquid glucose, magnesium aluminum silicate, maltodextrin, methylcellulose, polymethacrylates, povidone (e.g. Kollidon®, Plasdone®, pregelatinized starch, sodium alginate, and starch.

The dissolution rate of compacted solid compositions may be increased by the addition of a disintegrant to the compositions. Suitable disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of non-compacted solid compositions and to improve the accuracy of dosing. Excipients that may function as glidants include colloidal silicon dioxide, magnesium trisilicate, magnesium stearate, powdered cellulose, starch, talc, and tribasic calcium phosphate.

When a dosage form such as a tablet is made by the compaction of a powdered composition, the composition is subjected to pressure from a punch and dye. Some excipients and active ingredients tend to adhere to the surfaces of the punch and dye, which can cause the product to have pitting and other surface irregularities. A lubricant can be added to the compositions to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate. Preferably, said lubricant is present in between 0.25 and 1% (w/w) by weight.

Flavoring agents and flavor enhancers make the dosage form more palatable to the patient. Common flavoring agents and flavor enhancers for pharmaceutical products that may be included in the compositions as described herein include maltol, vanillin, ethyl vanillin, menthol, citric acid, fumaric acid, ethyl maltol and tartaric acid.

Solid compositions may also be dyed using any pharmaceutically acceptable colorant to improve their appearance and/or facilitate, patient identification of the product and unit dosage level.

In an embodiment and specifically when the compositions are intended for agricultural use, said excipients are agricultural compatible excipients. Said “agricultural compatible carriers” or “agricultural compatible excipients” which can be regarded as a vehicle, is generally inert and it must be acceptable in agriculture. Thus, the phrase “agriculturally compatible” denotes a substance that can be used routinely under field conditions without interfering with growers' planting equipment, and without adversely influencing crop development or the desired ecological balance in a cultivated area.

The agriculturally compatible carriers or excipients can be solid. Solid carriers or excipients can include but are not limited to clays, natural or synthetic silicates, silica, resins, waxes, solid fertilizers, a polymer, a granular mass, perlite, a perlite granule, peat, a peat pellet, soil, vermiculite, charcoal, sugar factory carbonation press mud, rice husk, carboxymethyl cellulose, fine sand, calcium carbonate, flour, alum, a starch, talc, polyvinyl pyrrolidone, or a combination thereof. The agriculturally compatible carrier or excipient can be a liquid. Liquid carriers or excipients can include but are not limited to water, alcohols, ketones, petroleum fractions, oils, aromatic or paraffinic hydrocarbons, chlorinated hydrocarbons, liquefied gases or a combination thereof. More particularly, the agriculturally compatible carriers or excipients can include a dispersant, a surfactant, an additive, a thickener, an anti-caking agent, residue breakdown, a composting formulation, a granular application, diatomaceous earth, a coloring agent, a stabilizer, a preservative, a polymer, a coating or a combination thereof. A person skilled in the art can readily determine the appropriate carrier or excipient to be used taking into consideration factors such as a particular compound, plant to which the inoculum is to be applied, type of soil, climate conditions, whether the inoculum is in liquid, solid or powder form, and the like. The additive can comprise an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutant dioate, a benzene acetonitrile derivative, a proteinaceous material, or a combination thereof. The proteinaceous material can include a milk product, wheat flour, soybean meal, blood, albumin, gelatin, or a combination thereof. The thickener can comprise a long chain alkylsulfonate of polyethylene glycol, polyoxyethylene oleate or a combination thereof. The surfactant can contain a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcolhol, an alkyl phosphate, or a combination thereof. The anti-caking agent can include a sodium salt such as a sodium sulfite, a sodium sulfate, a sodium salt of monomethyl naphthalene sulfonate, or a combination thereof; or a calcium salt such as calcium carbonate, diatomaceous earth, or a combination thereof.

In an embodiment, said compositions further comprises one or more of the following: water, other nutritive substance, weak acid, vegetable oil, essential oil, metabolism stimulant, emulsifying agent, viscosity agent, tinting material, suspending agent, dispersion agent, preservative, complexing agent, stabilizer, carrier, vehicle or wetting agent, or any combination thereof. In an embodiment, said compositions further comprises at least one oil, surfactant and polymer.

The compositions may be manufactured in a conventional manner. In certain embodiments, the compositions provided herein includes one or more preservatives to inhibit microbial activity. Suitable preservatives include mercury-containing substances such as merfen and thiomersal; stabilized chlorine dioxide; and quaternary ammonium such compounds as benzalkonium chloride, cetyltrimethylammonium bromide, and cetylpyridinium chloride.

The peptides as described herein can be formulated in the compositions at an amount of between 0.1 and 50 μg/ml, preferably between 0.1 and 45 μg/ml, preferably between 0.1 and 40 μg/ml, preferably between 0.1 and 35 μg/ml, preferably between 0.1 and 30 μg/ml, preferably between 0.1 and 25 μg/ml, preferably between 0.1 and 20 μg/ml, preferably between 0.1 and 15 μg/ml, preferably between 0.1 and 10 μg/ml, preferably between 0.1 and 9 μg/ml, preferably between 0.1 and 8 μg/ml, preferably between 0.1 and 7 μg/ml, preferably between 0.1 and 6 μg/ml, preferably between 0.1 and 5 μg/ml, preferably between 0.1 and 4 μg/ml, preferably between 0.1 and 3 μg/ml, preferably between 0.1 and 2 μg/ml, preferably between 0.1 and 1 μg/ml, preferably between 0.1 and 0.9 μg/ml, preferably between 0.1 and 0.8 μg/ml, preferably between 0.1 and 0.7 μg/ml, preferably between 0.1 and 0.6 μg/ml, preferably between 0.1 and 0.5 μg/ml or preferably between 0.1 and 0.4 μg/ml.

Alternatively, the peptides can be formulated in the compositions at an amount of between 0.2 and 50 μg/ml, preferably between 0.3 and 50 μg/ml, preferably between 0.4 and 50 μg/ml, preferably between 0.5 and 50 μg/ml, preferably between 0.6 and 50 μg/ml, preferably between 0.7 and 50 μg/ml, preferably between 0.8 and 50 μg/ml, preferably between 0.9 and 50 μg/ml, preferably between 1 and 50 μg/ml, preferably between 2 and 50 μg/ml, preferably between 3 and 50 μg/ml, preferably between 4 and 50 μg/ml, preferably between 5 and 50 μg/ml, preferably between 6 and 50 μg/ml, preferably between 7 and 50 μg/ml, preferably between 8 and 50 μg/ml, preferably between 9 and 50 μg/ml, preferably between 10 and 50 μg/ml, preferably between 15 and 50 μg/ml, preferably between 0.5 and 20 μg/ml, preferably between 25 and 50 μg/ml, preferably between 30 and 50 μg/ml, preferably between 35 and 50 μg/ml, preferably between 40 and 50 μg/ml, preferably between 45 and 50 μg/ml,

In another embodiment, the compositions contain at least 0.1% by weight of the peptide, more preferably at least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% by weight of peptide.

In a third aspect, the peptides or compositions as described herein are suitable for therapeutic use.

In an embodiment, the peptide or composition for therapeutic use comprises a peptide exhibiting at least 95% sequence identity to SEQ ID N° 1 or SEQ ID N° 2 or that differs with maximally 5 amino acids from the sequence according to SEQ ID N° 1 or SEQ ID N° 2.

In a further embodiment, said peptide comprises a signal peptide having a sequence identity of at least 95% to SEQ ID N° 3 or has a sequence according to SEQ ID N° 3.

“Therapeutic use” as described herein refers to the use of the peptides or the compositions thereof for ameliorating the symptoms of a disease in a human or non-human animal. In practicing the methods of treatment or use provided herein, a therapeutically effective amount of pharmaceutical composition described herein is administered to a subject such as a mammal or non-mammal having a disease, disorder, or condition to be treated. In some embodiments, the subject is a human. A therapeutically effective amount may vary widely depending on the severity of the disease, the age and relative health of the subject, the potency of the therapeutic agent used, and other factors. The therapeutic agents, and in some cases, compositions described herein, may be used singly or in combination with one or more therapeutic agents as components of mixtures.

In an embodiment, the peptides as described herein are used in pharmaceutical compositions for treatment or prevention of infections caused by a broad range of microorganisms including Gram-positive, Gram-negative bacteria, and/or fungi, preferably hyphal fungi or yeasts in a subject in need thereof. Said subject may be a human or animal. Said pharmaceutical compositions may contain a therapeutically effective amount of the antimicrobial peptide and a suitable carrier.

In an embodiment, the peptide or composition for use in the treatment of bacterial and/or fungal infections in a subject in need thereof comprises a peptide exhibiting at least 95% sequence identity to SEQ ID N° 1 or SEQ ID N° 2 or that differs with maximally 5 amino acids from the sequence according to SEQ ID N° 1 or SEQ ID N° 2.

In a further embodiment, said peptide comprises a signal peptide having a sequence identity of at least 95% to SEQ ID N° 3 or has a sequence according to SEQ ID N° 3.

The peptides or compositions described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but are not limited to, aqueous liquid dispersions, self-emulsifying dispersions, solid solutions, liposomal dispersions, aerosols, solid dosage forms, powders, immediate-release formulations, controlled release formulations, fast melt formulations, tablets, capsules, pills, delayed-release formulations, extended-release formulations, pulsatile release formulations, multi-particulate formulations, and mixed immediate and controlled release formulations.

In an embodiment, the peptides or compositions as described herein may be added to animal feed to reduce potential microbial infections in livestock. In another embodiment, the peptides or compositions described herein are formulated for oral, topical, or parenteral administration to treat microbial infections in veterinary medicine.

In another embodiment, the peptides or compositions as described herein are suitable for use in crop protection, as a feed or food additive, for pharmaceutical use, as a preservative and/or as a decontaminant.

In an embodiment, said peptide or peptide composition comprises a peptide exhibiting at least 95% sequence identity to SEQ ID N° 1 or SEQ ID N° 2 or that differs with maximally 5 amino acids from the sequence according to SEQ ID N° 1 or SEQ ID N° 2.

In a further embodiment, said peptide comprises a signal peptide having a sequence identity of at least 95% to SEQ ID N° 3 or has a sequence according to SEQ ID N° 3.

When used for crop protection, the peptides or the compositions as described herein are formulated with an agricultural compatible carrier or agricultural compatible excipient according to the embodiments above described. The person skilled in the art will determine the right dose, form, and method of application of said composition, in the function of the treated crop and the pathogen targeted.

In an embodiment, the peptides or compositions described herein are incorporated in feed or food additives to prevent pathogen spreading via feed and food. It is known that feed may carry pathogens that are detrimental to animal health and welfare. Antiseptic products such as formaldehyde are commonly used to disinfect the feed but these products can, in turn, exert negative effects. The antimicrobial peptides as described herein are a safe alternative.

In another embodiment, the peptides or the compositions as described herein are suitable to be used as a decontaminant or disinfectant of surfaces, tools, instruments, devices, objects, or body parts. In an embodiment, such devices or objects include, but are not limited to, linens, cloth, plastics, latex fabrics, natural rubbers, implantable devices, surfaces, or storage containers. In a particular embodiment, the peptides or the compositions as disclosed herein are incorporated in cleaning agents, detergents, soaps, or sprays.

In certain embodiments, the peptides as described herein can be incorporated into various health care products as well, particularly cosmetical products. For example, the peptides can be incorporated into toothpaste, mouthwash, shampoo, soap, cream, or antiperspirant to reduce or prevent microbial colonization or re-establishment of the oral cavity or skin.

In another embodiment, the peptides and compositions as described herein may be used as a food, feed, cosmetics, or pharmaceutical preservative or in treating food products to control, reduce, or eliminate potential pathogens or contaminants.

In another embodiment, the peptides or compositions as described herein are used for controlling a pathogen in a plant, preferably in a crop wherein said pathogens are Gram-positive and Gram-negative bacteria and/or fungi, preferably hyphal fungi or yeasts. Said plant can be a monocotyledonous or a dicotyledonous plant, including fodder or forage legumes, ornamental plants, food crops, trees or shrubs. In an embodiment, said plant is preferably a crop. Preferably, said plant belongs to the group of Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp., Averrhoa carambola, Bambusa spp., Benincasa hispida, Bertholletia excelsea, Beta vulgaris, Brassica spp., Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus spp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis spp., Eleusine coracana, Eragrostis tef, Erianthus spp., Eriobotrya japonica, Eucalyptus spp., Eugenia uniflora, Fagopyrum spp., Fagus spp., Festuca arundinacea, Ficus carica, Fortunella spp., Fragaria spp., Ginkgo biloba, Glycine spp., Gossypium hirsutum, Helianthus spp., Hemerocallis fulva, Hibiscus spp., Hordeum spp., Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp., Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp., Panicum miliaceum, Panicum virgatum, Passiflora edulis, Pastinaca sativa, Pennisetum spp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix spp., Sambucus spp., Secale cereale, Sesamum spp., Sinapis spp., Solanum spp., Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp., Tropaeolum minus, Tropaeolum majus, Vaccinium spp., Vicia spp., Vigna spp., Viola odorata, Vitis spp., Zea mays, Zizania palustris, Ziziphus spp.; including the progenies and hybrids between the above.

The peptides or compositions as described herein are suitable to be introduced to a plant, a plant part or a substrate comprising or hosting said plant, thereby conferring pathogen resistance or pathogen control to said plant. Said introduction may be artificial.

Methods for introducing the peptides or the compositions thereof to plants may include: treating the plant and/or a plant part and/or growth medium wherein said plant is grown, inoculation of the seeds, coating the seeds, direct inoculation of plants or plant parts, spraying or wetting plants or plant parts (eg. Ears). An appropriate method may be chosen depending on the type of plant to which the peptide or composition is to be introduced.

As another of further non-limiting example, the peptides or compositions as described herein may be applied in the form of coatings. The coating may be applied to the seed by spraying on the seeds or soaking said seeds in a solution containing the peptide or composition. In another example, for coating seeds with a solution as described in the previous example, a binding agent may be added, such as a binding agent comprising carbide (calcium carbonate).

In embodiments, the coating may be applied to a naked and untreated plant part. In other embodiments, the coating may be applied as an overcoat to a previously treated plant part. Seed coatings are particularly preferred in the treatment of soil-borne fungal diseases. In embodiments, the seed coating may be applied to a naked and untreated seed. In other embodiments, the seed coating may be applied as a seed overcoat to a previously treated seed.

In an embodiment, the peptides or compositions as described herein may be applied to the soil or any other substrate in which said plant grows in order to remove pests and/or pathogens from said substrate.

Inoculating the substrate comprising or hosting said plant or plant part can be performed, by way of example and without the intention to be limiting, using a powder, a granule, a pellet, a plug, or a soil drench that is applied to the substrate. Inoculation could also be performed by a liquid application, such as a foliar spray or liquid composition. The application may be applied to a growing plant or to the substrate. Plants, in particular agricultural plants, can be grown in the substrate. In one embodiment, said substrate is soil, sand, gravel, polysaccharide, mulch, compost, peat moss, straw, logs, clay, or a combination thereof. In another embodiment, the substrate can also include a hydroculture system or an in vitro culture system. In some embodiments, a combination of different application methods as described herein is applied.

In an embodiment, the peptides or the compositions as described herein are suitable for use in treating or preventing pathogenic infections wherein the pathogens are selected from the group consisting of Gram-positive bacteria, Gram-negative bacteria, and/or fungi as described in any of the previous embodiments.

The invention as disclosed herein also relates to a plant seed coated with the peptide or composition described in any of the previous embodiments.

In another aspect, the invention relates to methods for obtaining the peptides as disclosed herein, wherein said peptides are recombinant peptides, synthetic peptides, or are extracted or precipitated from Theobroma cacao.

In an embodiment, the methods as disclosed herein, are used for obtaining a peptide exhibiting at least 95% sequence identity to SEQ ID N° 1 or SEQ ID N° 2 or that differs with maximally 5 amino acids from the sequence according to SEQ ID N° 1 or SEQ ID N° 2 and wherein said peptide is a recombinant peptide, a synthetic peptide, or is extracted or precipitated from Theobroma cacao.

It was found that the variety CCN-51 Theobroma cacao has high levels of the peptide of the invention and thus it can be economically purified from CCN-51 cocoa beans using the suitable extraction and precipitation technique, followed by optimized chromatography techniques and other methods known in the prior art. Thus, the present invention offers a solution for a direct valorization of the CCN-51 cocoa beans, particularly non-fermented CCN-51 cocoa beans for isolation of a peptide that exhibits antimicrobial activity.

In a preferred embodiment, non-fermented and/or fermented and dried cacao beans are used for the extraction of the peptides as disclosed herein. In another embodiment, any type of Theobroma cacao tissue/organs is extracted to obtain the peptide of the present invention. It should be understood that cacao pods and/or cacao bean hulls, leaves, stems, and roots can be used as plant material for the extraction of the peptide of the invention without departing from the scope of the present invention. In another embodiment, the peptides as disclosed herein can be extracted or precipitated from any processed Theobroma cacao material.

In an embodiment, the cacao plant material is collected from selected cacao growing farms and used to isolate the peptides as described herein. Preferably, the samples are stored at 4° C. for further processing. Alternatively, the samples are stored at −80° C. prior to processing.

For the extraction of peptide non-fermented, well-dried beans may be used. Mucilaginous pulp from the non-fermented beans is typically removed, preferably wiped off. Seed coats are peeled off with a sterile scalpel, forceps and/or any other suitable apparatus.

Beans are rich in lipids and in a preferred embodiment, cocoa beans are defatted before peptide extraction. In one embodiment, the ground cocoa beans can be defatted in acetone, the residue powdered beans dried, and then extracted in an aqueous buffer. Enzyme inactivation is reduced by slowly adding very cold acetone during extraction

In an embodiment, said cacao beans are subjected to deshelling before peptide extraction. Deshelled bean materials have shown the best extractability of the peptide as compared to other cocoa parts, such as, but not limited to shells and pulp.

The cocoa beans are finely ground using a suitable mill, grinder, or a mortar and pestle, whereby said apparatuses are preferably pre-chilled, in a cold atmosphere, a freezing chamber, or cooled by liquid nitrogen. The ground powder is typically stored at a temperature not exceeding 4° C., preferably not exceeding 0° C., most preferably at −20° C. for further processing

In another embodiment, frozen plant material is used for the extraction of the peptides as described herein. All operations are carried out at 0° to 4° C. (i.e., in a cold room, or on ice). The extraction procedure is performed rapidly to minimize exposure of proteins of interest to potentially damaging compounds and enzymes released upon cell breakage.

In an embodiment, cacao beans are ground without letting the liquid nitrogen come into contact with the tissue. To ensure that the sample stays frozen, the mortar/mill and/or grinder may be placed in a shallow pool of liquid nitrogen (e.g., in the lid of a standard polystyrene box). Fresh material may be ground directly in the ice-cold buffer. In one embodiment, a tissue homogenizer is used for grinding non-fermented, fresh cocoa beans. In another embodiment, cocoa beans are ground in a blender or a mortar and pestle with the addition of acid-washed sand. Foaming can be a problem when blending, and some measure of control may be achieved by adding a few drops of n-octanol or any other suitable organic solvent known in the art.

In an embodiment, an extraction buffer of a pH of preferably 8 is used for the extraction. Said buffer maintains the stability of the isolated peptides from both a pH and an ionic strength standpoint. Some of the non-limiting examples of buffers used include N-2-hydroxyethylpiperazine N-2-ethanesulfonic acid (HEPES), phosphate, 2-(N-morpholino)-ethanesulfonic acid (MES), trisaminomethane (TRIS), TAE buffer (Tris base, acetic acid, and EDTA), TBE (Tris base, boric acid, and EDTA) or any mixture thereof in acetone, phenol or any other suitable solvent or a mixture thereof. It should be understood by a skilled person that a choice and a type of buffer are optimized depending on the cocoa variety, the use of fermented or non-fermented beans in the extraction process. Any suitable buffer commonly used in the state of the art can be employed to extract the peptides, without departing from the scope of the present invention. The volume of extraction buffer used per unit weight of plant material and/or cocoa beans depends on the plant material type and the peptide concentration. According to one embodiment of the invention, a better extraction is achieved by using a higher ratio of buffer volume to plant material and/or cocoa beans weight (e.g., 10:1).

In an embodiment, in an extraction process of the peptides as described herein, a sample extract is first concentrated. This can be done by precipitation, a suitable chromatography technique, or any other method known in the art. In an embodiment, 10% to 20% glycerol (v/v) is added to an extraction buffer to ensure the stability of the peptides.

In an embodiment, an antioxidant is added to the extraction buffer in order to prevent oxidations of the peptides, to maintain the reduced state of free sulfhydryl groups, and to reduce oxidation of other components, such as phenolics. Some non-limiting examples of such antioxidants include dithiothreitol (DTT), β-mercaptoethanol, or ascorbic acid and in some circumstances, if necessary stronger reducing agents such as sodium dithionite. In another embodiment, more than one reducing agent can be used, such as but not limited to a combination of DTT and β-mercaptoethanol.

In an embodiment, detergents are added to the buffer, in order to enhance disruption of the membranes and solubilization of membrane proteins. Some non-limiting examples include sodium dodecyl sulfate (for instance between 0.1 to 1.0%), Tween 80 (for instance from 0.1% to 1%), Triton X-100 (for instance from 0.1% to 1%), 3-[(3-cholamidopropyl)dimethylammonio]-2-hydroxy-1-propanesulfo-nate (CHAPS) or octyl glycosides, or any mixture thereof. It should be understood by a skilled person that initial empirical testing is needed to determine optimal combinations of surfactants for maximizing the concentration of obtained vicilin (precursor) peptides in the extract.

It should be understood that protective agents, such as but not limited to polyvinylpolypyrrolidone (PVPP), soluble polyvinyl pyrrolidone (PVP), and/or polyethylene glycol may be used to prevent binding of polyphenols such as tannins to the antimicrobial peptides of the invention.

Additionally, any proteinase inhibitor can be added to a buffer during the extraction of the antimicrobial peptides for controlling the degradation of proteins following extraction. Some non-limiting examples of said proteinase inhibitors are phenylmethylsulfonyl fluoride (PMSF), bovine pancreatic trypsin inhibitor (also known as aprotinin), 4-(2-aminoethyl)-benzenesulfonyl-fluoride hydrochloride, ethylenediaminetetraacetic acid (EDTA), 1,10-phenanthroline, or 2,2-bipyridyl, trans-epoxysuccinylL-leucyl-amido-(4-guanidino) butane, antipain and leupeptin, pepstatin, aspartic proteinases, commercially available proteinase inhibitors (e.g., those provided in the form of tablets by Roche and as solutions by Sigma) containing a cocktail of these inhibitors tailored for plants, and the like.

In a particularly preferred embodiment, Tris-HCl buffer with added dithiothreitol (DTT) as an antioxidant and sodium dodecyl sulfate (SDS) as an anionic surfactant in an alkaline environment and optionally in presence of a proteinase inhibitor cocktail is used for the extraction.

The extraction/incubation time is optimized based on the extraction mixture and the concentration of antimicrobial peptides in the plant material and can vary from 15 min to 48 h, preferably not less than 30 min, most preferably not less than 1 h, and preferably not more than 5 h, most preferably not more than 3 h. Extraction temperature should preferably not exceed 40° C., preferably should not exceed room temperature (23° C.), and most preferably is done at a temperature not exceeding 15° C.

In an embodiment, the peptides as disclosed herein may be isolated from cocoa beans. Said cocoa beans may be fermented or non-fermented. In another or further embodiment, they may be dried and/or non-dried cocoa beans. In a preferred embodiment, the peptides as described herein may be isolated from non-fermented, dried cocoa beans. In an embodiment, the cocoa beans are crushed, the shells are removed and the nibs are pressed in an expeller. Thereby, a mixture of butter and slurry is obtained. Said slurry is used for peptide extraction in some embodiments.

In the embodiments whereby, the other cacao plant parts are used to extract the peptide, numerous protectants, such as buffers, antioxidants, proteases inhibitors, and like are used to stabilize the proteins in the extracts. The amount of extraction solvents is kept to a reasonable minimum, to avoid dilution of protein extracts. Cocoa fruits are acidic, with high levels of polyphenolics, and need high buffer concentrations to maintain pH.

The protein extracts obtained by an extraction step according to the present invention are preferably separated using sedimentation, centrifugation, filtration, or any other commonly used technique to obtain protein extracts freed from the residual plant material. Filtration through a cloth, such as, but not limited to Miracloth and the like, can be used. In another embodiment, a nylon mesh filter is preferred as it is very strong and can be washed and reused.

In an embodiment, the filtrate is run through a large flash chromatography column. In this way, various low-molecular-weight polyphenols that bind to the proteins during large-scale production, can be separated from proteins.

For the preparation of small tissue samples for crude assays, the extract can be desalted using small gel-filtration columns, such as but not limited to PD-10 or NAP columns, Amersham Biosciences, and the like.

In another embodiment, the peptides as described herein are isolated from cocoa beans by isoelectric precipitation. Typically, this precipitation is done once the undesirable fractions, such as carbohydrates, fat, and/or oil are removed by any suitable method known in the art. This step concentrates the protein in the remaining portion of plant material.

According to the present invention, said portions are dissolved in a buffer of a particular pH, and the pH is lowered to the isoelectric point of the proteins so that they can get precipitated. In a preferred embodiment, the proteins from the extract are precipitated using an ammonium sulfate solution, preferably an ammonium sulfate solution in the concentration of at least 10 wt. % and at most 20 wt. %, most preferably an ammonium sulfate solution of a concentration between 12 to 14 wt. %. The proteins are collected from the protein precipitates by centrifugation.

In a further preferred embodiment, the precipitated/extracted proteins are further separated by electrophoresis. In a particularly preferred embodiment, said electrophoretic separation is two-dimensional electrophoresis. Said electrophoresis is preferably done in a polyacrylamide gel. In an embodiment, said electrophoresis is done in a 15% SDS-PAGE gel. The electrophoresis enables high purification of the peptides, eliminating the disadvantages of non-electrophoretic techniques that are labor-intensive and costly. The vertical field “separates” the proteins according to their mobility in the field, which is predominantly based on their molecular weight.

In a further embodiment, the separation is followed by electro-elution and the like, whereby the peptides are drawn to the surface of a gel thereby enabling extraction and subsequent analysis of said peptide. In an embodiment, the bands corresponding to the peptides are excised from the electrophoresis gel, washed in Tris-EDTA, extracted via sonication and centrifugation.

In a further preferred embodiment, the peptide of interest is dried by lyophilization or vacuum centrifuged. Alternatively, the peptides can be precipitated with a suitable agent. The lyophilized and/or precipitated peptide is optionally re-suspended in distilled water and/or any other suitable solvent and used for further analyses.

In another embodiment, the extracted or precipitated peptides are concentrated. Preferably, said peptides are concentrated by standard salt or organic solvent precipitation. Alternatively, the peptides are concentrated by dialysis against a volatile buffer (e.g., ammonium carbonate), by using a filter-based concentrator, or by ion-exchange chromatography. It should be known by a skilled person that the concentration of the extracts/precipitates is a slow and critical process and thus special attention should be taken that the peptides are not exposed to polyphenols and proteinases during these processes.

The quantification of the peptide content is done by spectrophotometric methods such as the Lowry method or Bradford assay, or any other method known in the art. In another embodiment, the quantification of the peptide is done by any suitable chromatographic method known in the art. In a preferred embodiment, an LC-MS method is used for the quantification of the peptide content. In a preferred embodiment, MALDI-TOF analyses are conducted on fragments generated via tryptic digestion of the proteins obtained from cacao extracts. In one embodiment, the data obtained from MALDI-MS tryptic digest analyses is further corroborated by preparing and performing immunochemical assays to detect specific differences between samples.

In a further preferred embodiment, said peptide is substantially free of other peptides. The term “substantially free”, as used herein, means at least 95 wt. % free of other peptides; preferably at least 99 wt. % free of other peptides. In a preferred embodiment, said peptide is present in a crystal and/or a solid form.

In another aspect, the present invention relates to a vector comprising a coding sequence for a peptide having a sequence identity of at least 95% with SEQ ID N° 1 or SEQ ID N° 2 or that differs with maximally 5 amino acids from the sequence according to SEQ ID N° 1 or SEQ ID N° 2, and optionally comprising a sequence for an N terminal signal peptide, said signal peptide sequence having a sequence identity of 95% to SEQ ID N° 3, or is a sequence according to SEQ ID N° 3, wherein said vector is designed to allow expression of said peptide in an expression system. In a preferred embodiment, the vector comprises a coding sequence of a peptide having 96%, more preferably 97%, more preferably 98%, more preferably 99% or more preferably 100% identity to SEQ ID N° 1 or SEQ ID N° 2. In an embodiment said vector comprises a purification tag as disclosed in a previous embodiment.

In an embodiment, the DNA extracted from cacao beans is used to amplify the coding sequence of the peptide, and is cloned in a suitable expression vector such as any plasmid or virus designed for gene expression in cells.

The peptides as described herein can thus be expressed in any suitable system for the purpose of producing the peptide for further use. Suitable hosts for the expression of the protein include Escherichia coli, fungal cells, insect cells, mammalian cells, and plants. Standard methods for expressing proteins in such hosts are described in a variety of texts including section 16 (Protein Expression) of Current Protocols in Molecular Biology (supra).

In general, DNA encoding the amino acid sequence of interest is contained in an expression vector, in some cases linked in-frame at the 5′ or 3′ end to another coding sequence so as to encode a peptide of at least 95% of sequence identity to SEQ ID N°1 or SEQ ID N°2, and optionally comprising at the N-terminus a SEQ ID N°3. The total coding sequence is operably linked to a promoter such that the promoter drives the expression of the coding sequence. The coding sequence is also referred to herein as the “target gene”.

According to an embodiment, the promoter is either a promoter native to the microorganism (for example the Escherichia coli trpE promoter), a synthetic promoter such as the Tac promoter, or a promoter obtainable from a heterologous organism, for example, a virus, a bacterium or a bacteriophage such as a phage A or T7 which is capable of functioning in the microorganism. The promoter may be constitutive or, more preferably, inducible. The expression vector may also contain a selectable marker gene, which may be an antibiotic resistance gene such as an ampicillin, tetracycline, chloramphenicol, or kanamycin resistance gene.

Many promoter systems are available, often from commercial sources, which are suitable for the expression of peptides of the invention in Escherichia coli. For example, the PBAD promoter from the araBAD (arabinose) operon has advantageous induction properties, being inducible 1,200-fold over background (Guzman et al., 1995). Others include P_{LAC}, P_{TAC}, P_{TRC}, PL and PR. The P_{TAC} promoter is a hybrid derived from the trp and lac promoters of Escherichia coli, and is one of the most potent Escherichia coli-based promoter systems known. It is inducible by IPTG, as with the lac promoter.

The preferred host cells of the present invention are of Escherichia coli M15 host strain. In another preferred embodiment, Pichia pastoris are used as host cells for the expression of the peptides of the invention.

In a further preferred embodiment, the selection of clones is done and over-expressed peptides are extracted via their affinity tags, using Ni-NTA agarose. The recombinant peptides are separated and purified using the same methods applied for the peptides isolated from natural sources in a previous embodiment.

Expression of the new peptide in a genetically engineered cell will typically result in a product having a three-dimensional structure identical to naturally occurring peptide, which is isolated from cacao plant material. This three-dimensional structure includes correctly formed intramolecular disulfide linkages between cysteine residues. However, even if the protein is chemically synthesized, methods are known in the art for further processing of the protein to break undesirable disulfide bridges and form the bridges between the desired cysteine residues to give the desired three-dimensional structure should this be necessary.

In another embodiment of the invention, said peptide can be obtained by chemical synthesis using the known peptide-synthetizing techniques, such as solid-phase syntheses on instruments such as room-temperature peptide synthesizers, microwave peptide synthesizers, parallel peptide synthesizers and the like.

In one embodiment of the invention, it is possible to express the peptide as described herein in transgenic plants. Consequently, the current invention also relates to transgenic plants expressing one or more peptides as described herein.

In an embodiment, the transgenic plant expresses a peptide exhibiting at least 95% sequence identity to SEQ ID N° 1 or SEQ ID N° 2 or that differs with maximally 5 amino acids from the sequence according to SEQ ID N° 1 or SEQ ID N° 2. In a further embodiment, said peptide comprises a sequence for an N terminal signal peptide, said signal peptide sequence having a sequence identity of 95% to SEQ ID N° 3.

In a further embodiment, the transgenic plant expresses a peptide having 96%, more preferably 97%, more preferably 98%, more preferably 99% or more preferably 100% identity to SEQ ID N° 1 or SEQ ID N° 2.

In an embodiment said peptide comprises a purification tag as disclosed in a previous embodiment.

According to the present invention, plant cells can be transformed with DNA constructs according to a variety of known genetic engineering methods (Agrobacterium tumefaciens transformation, Ti plasmids, electroporation, micro-injections, micro-projectile gun, PEG-mediated transformation and the like). A genetic engineering method as used herein may be understood as any methods used to introduce foreign DNA sequences into plant cells, in order to regenerate genetically modified plants that express desired traits or characteristics.

In one embodiment of the invention, DNA sequences encoding a peptide of at least 95% sequence identity to SEQ ID N°1 or SEQ ID N°2, and optionally comprising an SEQ ID N°3 at the N-terminus, as well as DNA coding for homologs from other plant species, can be used in conjunction with a DNA sequence encoding a preprotein from which the mature protein is produced. This preprotein contains a native peptide sequence, which will target the protein to a particular cell compartment (e.g., the apoplast or the vacuole). These coding sequences can be ligated to a plant promoter sequence that will ensure strong expression in plant cells. This promoter sequence might ensure strong constitutive expression of the protein in most or all plant cells, it may be a promoter that ensures expression in specific tissues or cells that are susceptible to microbial infection and it may also be a promoter that ensures strong induction of expression during the infection process. These types of gene cassettes will also include a transcription termination and polyadenylation sequence 3′ of the antimicrobial protein-coding region to ensure efficient production and stabilization of the mRNA encoding the antimicrobial proteins. It is possible that the efficient expression of the antimicrobial peptide disclosed herein might be facilitated by the inclusion of their individual DNA sequences into a sequence encoding a much larger peptide.

Gene cassettes encoding the peptides are expressed in plant cells using methods known in the art. Firstly, the gene cassettes can be ligated into binary vectors carrying: i) left and right border sequences that flank the T-DNA of the Agrobacterium tumefaciens Ti plasmid; ii) a suitable selectable marker gene for the selection of antibiotic-resistant plant cells; iii) an origin of replication that functions in both Agrobacterium tumefaciens and Escherichia coli; and iv) antibiotic resistance genes that allow selection of plasmid-carrying cells of Agrobacterium tumefaciens and Escherichia coli. Without wishing to be limitative, the DNA sequence of the peptides disclosed herein can be cloned in any binary vectors known in the art, such as plasmids (pEXA128, pBR322, pUC19), bacteriophage (λ phage, M13 phage), cosmids, BACs or YACs. The binary vector carrying the DNA sequence of the peptides can be introduced by either heat shock, electroporation or triparental mating into Agrobacterium tumefaciens strains carrying disarmed Ti plasmids such as strains LBA4404, GV3101, and AGL1 or into Agrobacterium rhizogenes strains such as A4 or NCCP1885. These Agrobacterium strains can then be co-cultivated with suitable plant explants or intact plant tissue and the transformed plant cells and/or regenerants selected using antibiotic resistance. Alternatively, the binary vector carrying the DNA sequence of the peptides can be overexpressed in bacterial or fungal cells.

The present disclosure also relates to a method of obtaining a transgenic plant expressing a peptide exhibiting at least 95% sequence identity to SEQ ID N° 1 or SEQ ID N° 2 or that differs with maximally 5 amino acids from the sequence according to SEQ ID N° 1 or SEQ ID N° 2 and optionally comprising a sequence for an N terminal signal peptide, said signal peptide sequence having a sequence identity of 95% to SEQ ID N° 3.

The method comprises the steps of:
(a) optionally isolating or synthesizing a nucleic acid sequence encoding a peptide exhibiting at least 95% sequence identity to SEQ ID N° 1 or SEQ ID N° 2 or that differs with maximally 5 amino acids from the sequence according to SEQ ID N° 1 or SEQ ID N° 2;
and incorporating the nucleic acid sequence into a suitable expression vector;
(b) introducing the nucleic acid into at least a plant cell by a genetic engineering method to produce a transformed plant cell expressing said peptide;
(c) regenerating the transformed plant cell into whole transgenic plants; and
(d) selecting and identifying transgenic plants expressing said peptide using suitable screening methods.

In an embodiment of the method as disclosed herein, the nucleic acid sequence encoding for the peptide is isolated using any method known in the art, such as gene cloning methods. None limiting examples of gene cloning include PCR-based cloning and restriction enzyme-based cloning.

In another embodiment of the method disclosed herein, the DNA sequence of the peptides is introduced into the plant cell by biolistic bombardment. Biolistic bombardment involves introducing foreign DNA sequences into plant cells using a gene gun. The DNA sequence encoding the antimicrobial peptide as disclosed herein is attached to metal particles, such as gold particles, and then bombarded into the plant cells.

In yet another embodiment, the DNA sequence of the peptides is introduced into the plant cell by electroporation which involves the use of electrical pulses to create pores in the cell membrane, allowing the DNA sequence encoding the antimicrobial peptide to enter said cell. Alternatively the DNA sequence encoding the antimicrobial peptide may be directly injected into the plant cell (microinjection).

In yet another alternative embodiment the DNA sequence encoding said peptide is introduced into the plant cells by PEG-mediated transformation that involves the use of polyethylene glycol (PEG) to create temporary pores in the cell membrane, allowing the DNA sequence to enter the cell.

Any plant organ, tissue or explant may be used with the genetic engineering method in order to obtain the transgenic plant as disclosed herein. Non limiting examples include: individual cells, callus, seeds, leaf explants, stem explants, root explants, embryonic tissue, meristems or pollen.

Overall, the specific genetic engineering method and explant used to obtain the transgenic plant expressing the peptide as disclosed herein depend on factors such as the plant species, the efficiency of the transformation method, and the desired level of expression of the antimicrobial peptide.

In a further embodiment of the method as disclosed herein the transformed cell is regenerated into an entire plant expressing the peptide.

The transgenic plants expressing the peptide are identified and selected. This step is essential for eliminating the non-transformed plans. Antibiotic or herbicide resistance selection is one of the most commonly used selection methods for transgenic plants. The transgenic plants are transformed with a gene that confers resistance to an antibiotic or herbicide and are then grown on a medium containing said antibiotic or herbicide, which kills or inhibits the growth of the non-transformed plants. In some embodiments, the transgenic plants are transformed with a gene that encodes the reporter protein and can then be easily identified and selected based on the expression of the reporter gene.

In other embodiments, the transgenic plants are selected by nutritional selection. This method is based on the ability of the transgenic plants to grow on media lacking a specific nutrient that is essential for the growth of non-transformed plants. The transgenic plants are transformed with a gene that confers the ability to synthesize the missing nutrient.

Marker-free selection involves the use of selectable markers that can be removed from the transgenic plants after selection. This can be achieved using site-specific recombination systems, such as the Cre-lox system, which allows for the removal of the selectable marker gene without leaving any trace of the foreign DNA.

The invention is further described by the following non-limiting examples which further illustrate the invention, and are not intended to, nor should they be interpreted to limit the scope of the invention.

### EXAMPLES

### Example 1: Peptide Extraction, Purification, and Identification

Cocoa bean samples derived from cocoa hybrid CCN-51 were collected from small, traditional, and more than 30-year-old cocoa plantations in Ecuador. The samples were provided in non-fermented and fermented/dried form by Barry Callebaut AG. Non-fermented samples were shipped on dry ice and stored at −80° C. upon arrival while fermented dry samples were shipped and stored at 4° C. for further processing. Mucilaginous pulp from the non-fermented beans was wiped off and seed coats were peeled off with a sterile scalpel. The seed coats of fermented beans were removed using forceps. The beans were finely ground using Retsch Grindomix GM200 knife mill at 10,000 rpm. The ground powder was used directly (or stored at −20° C. for further processing).

From the finely ground powder, 250 mg were transferred to a 2-mL polypropylene microcentrifuge tube and suspended in 1 mL of protein extraction buffer (100-mM Tris HCl, 1% DTT, and 1% SDS; pH adjusted to 8.1; protease inhibitor cocktail according to manufacturer's instruction). The mixture was vortexed thoroughly for 60 s and incubated for 60 min with gentle shaking at 4° C., followed by centrifugation at 13,200 rpm for 20 min and at 4° C. in a benchtop 5810R centrifuge. The supernatant containing the protein fraction was gently transferred to a fresh cup using pipetting and stored at −20° C. for further analysis.

The content of extracted cocoa proteins was assessed using the method by Bradford MM disclosed in “Analytical Biochemistry” 1976; 72:248-54.

According to the Bradford method, to 10 μl diluted protein solution, 300 μl of Bradford reagent were added in a microtiter plate and briefly vortexed. After 10 min incubation at room temperature, absorbance was measured at 595 nm in triplicates. Extracted protein concentrations were quantified from a standard curve using bovine serum albumin in the concentration range 1-1500 μg/ml.

The extracted cocoa proteins and/or peptides were separated according to their molecular weight using SDS-PAGE. In short, 25 μl of the protein sample was mixed with 5 μl of 6× sample buffer containing bromophenol blue as tracking dye. The mixture was heated at 95° C. for 5 minutes and loaded on SDS-PAGE gels (83 mm×65 mm×1 mm) containing 12.5% or 15% (w/v) acrylamide. Electrophoresis was performed at 130 V for 90 min. After electrophoresis, the gel was either stained with Coomassie® Blue (45% (v/v) methanol, 10% acetic acid, 2.93×10⁻³ M Coomassie® Brilliant Blue G-250) for 20 min or blotted electrophoretically onto PVDF membranes as described previously by Towbin et al. in Proc. Natl. Acad. Sci. USA, 1979, 76, 4350-4354. The blots were treated with 5% (w/v) milk in PBST overnight at 4° C. Subsequently, the protein blots were probed with a polyclonal antibody that was diluted 1:2,000 in PBST containing 5% (w/v) milk-powder and incubated for 2 hours (h) at room temperature. Further, the blots were washed six times, 5 min each at room temperature under gentle shaking. Subsequently, the blots were incubated in HRP-conjugated goat anti-rabbit IgG antibody for 1 h at room temperature diluted 1:10,000 in PBST containing 5% (w/v) milk-powder. After incubation, the blots were washed six times with PBST, 5 min each at room temperature under gentle shaking. Finally, the indirectly bound HRP was detected by its enzymatic activity using luminol as substrate in the presence of hydrogen peroxide, as described by Mruk and Cheng in Spermatogenesis, 2011, 1, 121-122.

Before proceeding to two-dimensional electrophoresis, total protein extracts were purified using a Ready Prep™ 2D clean-up kit. For removal of interfering substances such as ionic detergents, lipids, or phenolic compounds, total protein extracts were purified using said kit as recommended by the manufacturer. After precipitation and washing in wash buffer provided with the kit, the protein sample was re-suspended in 130 μL of rehydration buffer (2 M thiourea, 6 M urea, 16.2×10⁻³ M CHAPS, 25.9×10⁻³ M DTT) and supplemented with ampholytes.

2D gel electrophoresis of cocoa proteins was performed by isoelectric focusing and subsequent SDS-PAGE. For this, 80 μg of protein were applied to immobilized pH gradient (IPG) strips (7 cm, pH 3-10) and soaked for 14 h at room temperature. Isoelectric focusing was carried out on a Bio-Rad Protean® i12™ IEF Cell (50 V, 70 min; 150 V, 20 min; 300 V, 15 min; gradient to 600 V, 10 min; 600 V, 15 min; gradient to 1,500 V, 10 min; 1,500 V, 30 min; gradient to 3,000 V, 20 min; 3,000 V, 210 min; pause on 50 V). Next, the IPG strips were equilibrated for 15 min in 6.48×10⁻² M DTT and 0.216 M iodoacetamide solution dissolved in equilibration buffer (6 M urea, 30% (w/v) glycerol, 69.2×10⁻³ M SDS in 0.05 M Tris-HCl buffer, pH 8.8) at room temperature. Molecular weight separation was conducted on a Bio-Rad Mini-Protean® Tetra System (110 V, 10 min; 130 V further on) via a 12.5 or 15% polyacrylamide gel. The molecular weight of the proteins was assessed by their visual mobilization in polyacrylamide gel and the predicted weight of the amino-acid sequence. After staining and de-staining, the resulting 2D gels were scanned in an Epson Scanner.

The scanned 2D gels were processed by ImageMaster 2D Platinum software 6.0 software (GE Healthcare). In doing so, automatic spot detection parameters were set for all samples to: Smooth=6, Saliency=100, Min Area=5. The detected non-protein spots, i.e., contaminating artifacts including streaks and dust particles, were removed during manual spot selection. Spot volumes were by default background-subtracted on a spot basis, by excluding the lowest tenth percentile pixel values on the spot boundary, from all other pixel values within the spot boundary. For matching the 2D gel set, a reference sample, termed master gel, was defined that enabled the identification of identical protein spots across different gels. The resulting match report lists the matching spots in the individual 2D gels with the corresponding spots in the master gel along with the respective normalized relative spot volume.

Protein spots of interest were excised from the 2D gels, chopped into small pieces, and washed twice for 15 min in 100 μL of 0.05 M ammonium bicarbonate buffer, containing 50% ACN (v/v). Gel pieces were dehydrated by the addition of 500 μL acetonitrile and incubated for 10 minutes at room temperature. After decanting and short air-drying, samples were supplemented with trypsin digestion buffer and incubated for 30 min at 55° C. as previously established. The samples were directly used for Matrix-Assisted Laser Desorption Ionization-TOF Mass Spectrometry (MALDI-TOF-MS) analyses.

For spectrometric identification of peptide patterns, the peptides were either reduced with 15 mM TCEP or used in the un-reduced form. The peptides were loaded on a Proshell 300SB-C8 column (1 mm internal diameter×75 mm, 5 μl particles) equipped with a narrow bore-guard column Zorbax 300SB-C8 (2.1 mm internal diameter×12.5 mm, 5 μm particles). Proteins were eluted from the column by a linear 5 min gradient from 95% solvent A (0.1% formic acid (FA), 0.05% trifluoroacetic acid (TFA) in water) to 90% solvent B (0.1% FA, 0.05% TFA in acetonitrile), followed by a 3 min wash at 90% solvent B, returning to 5% solvent B in 2 min for an equilibration of 2.5 min. The mass spectrometer was operated in MS mode at a resolution of 60000 (at m/z 400) in a mass range from 600 to 4000 m/z. An AGC setting of 1E7 was used, allowing to fill the trap for 1 s. Source settings were set at a SID of 15V, a capillary temperature of 325° C. at a voltage of 4.2 kV.

The recorded spectra were deconvoluted with the Xtract™ algorithm in the Freestyle software (Thermo fisher scientific, USA).

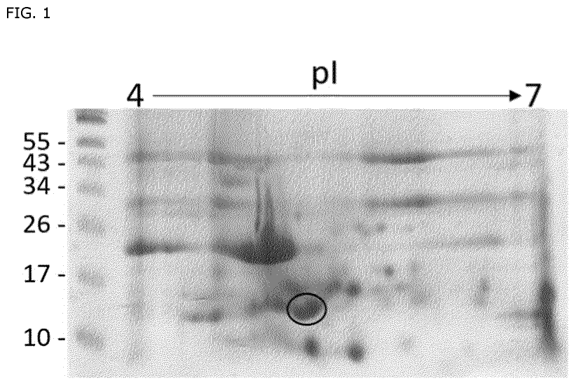

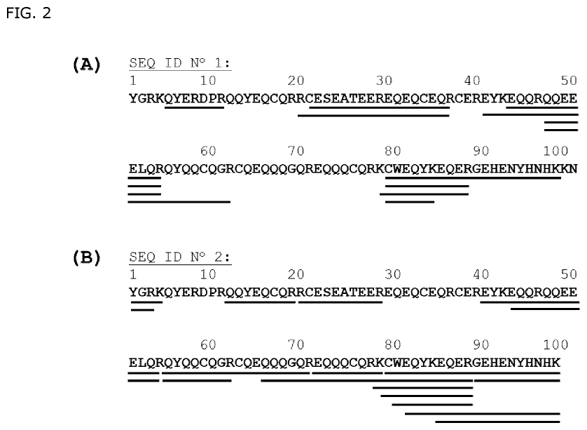

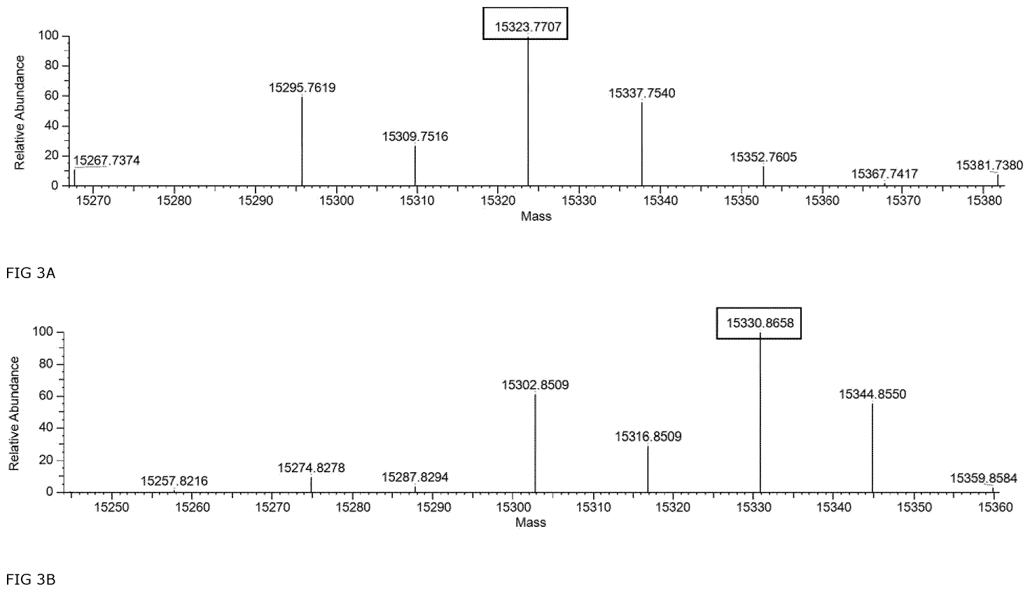

Results: The peptides as disclosed herein were identified in the total protein fraction isolated from beans of CCN-51 cocoa variety. After the 2D separation of the proteins isolated from cocoa beans a spot corresponding to 15 kDa was excised and trypsin-digested (FIG. 1). The digested peptides were identified using MALDI-TOF-MS) analyses (FIG. 2A). Recombinant peptide of SEQ ID N° 2 was separated by SDS polyacrylamide gel electrophoresis, excised from the gel, and subjected to trypsin digestion resulting in a MALDI-TOF-MS peptide finger print depicted in FIG. 2B. In both the reduced and non-reduced sample containing the peptides according to SEQ ID N° 2, masses of 15.3 kDa were detected using intact mass fingerprinting. The LC-MS results of the sample according to SEQ ID N° 2 are shown in FIG. 3.

### Example 2: Recombinant Peptide Production

Total DNA was extracted from embryos of the CCN-51 cocoa beans and the nucleotide sequence coding for the peptide was amplified using the primers Forward_BamH1-5′CGCGGATCCTATGGCAGAAAACAATAT3′ and Reverse_Kpn1-5′CGCGGTACCTTTGTGATTATGGTAATT3′. This resulted in an amplified fragment of 300 bp DNA (DNA of interest).

The amplified DNA fragment was ligated into the BamH1/Kpn1 sites of the cloning vector pEXA128. Both the plasmid caring the DNA of the peptide and the pQE30 overexpression plasmid were transformed into E. coli DH5α cells for amplification. The plasmids were extracted from overnight cultures, were digested with BamHI and KpnI at 37° C. for 2 hours and were separated on 1% agarose gels. Linearized pQE30 plasmids and inserts from the pEXA128 plasmids were extracted from the gels and inserts were ligated into the pQE30 plasmid. The pQE30 overexpression plasmid encodes the HIS tag and overexpression of the peptide of the invention will give rise to a fusion peptide consisting of the peptide and an N-terminal HIS tag. The resulting plasmids were transformed into E. coli M15 overexpression cells. Plasmids that were extracted from overnight cultures were sequenced and only clones that matched the expected sequences were used for overexpression. 16 ml overnight cultures were used to inoculate 800 ml liquid LB medium at 37° C. Once an OD₆₀₀ of between 0.6 and 0.8 was reached, IPTG was added to a final concentration of 1 mM to induce overexpression. The cells overexpressing the peptides containing the signal peptide were incubated for 24 hours at 20° C., and the cell overexpressing the peptides without the signal peptide for 5 hours at 37° C.

The recombinant peptide was purified and identified as described in Example 1.

### Example 3: Synthesis of the Peptides

The peptides of sequence identical to SEQ ID N° 1 and SEQ ID N° 2 were chemically synthesized by Seramun Diagnostica GmbH (Heidesee, Germany).

### Example 4: Minimum Inhibitory Concentration (MIC) Assays

MIC assays determine the lowest concentration at which a compound inhibits the growth of a microbial population. The antimicrobial potential of the peptides described herein was compared with the activity of two other AMPs, TcAMP1 and TcAMP2 as discussed in WO1998027805, and antibiotics or fungicides. Comparative MIC assays were carried out against several bacterial and fungal species (Table 1).

Peptides according to SEQ ID N° 1, SEQ ID N° 2, SEQ ID N° 4, SEQ ID N° 6, and SEQ ID N° 7 were serially diluted in order to create a concentration sequence ranging from 0.39 μg/ml to 303 μg/ml.

Pantoea, Staphylococcus, and Listeria were cultured in Trypticase Soy Broth (TSY) medium consisting of 1.7% casein peptone, 0.3% soy peptone, 0.25% glucose, 0.5% NaCl, 0.25% K₂HPO₄ and optionally 1.5% agar. Candida cells were cultured in Yeast Extract Peptone Dextrose (YPD) broth medium consisting of 1% yeast extract, 2% peptone, 2% glucose, and optionally 2% agar. Ceratocystis, Botrytis, Leptosphaeria, Mycosphaerella, Sclerotinia, and Verticillium were cultured in Potato Dextrose Agar (PDA) medium consisting of 2.4% Difco pre-mix broth and optionally 1.5% agar.

For the MIC assay bacteria and fungi were grown on agar plates with their respective media. Pre-cultures were prepared by inoculating either single bacterial colonies or small pieces of fungi in 5 ml of the respective liquid medium. The optical density (OD) at 600 nm was measured after one to five days and adjusted to an OD₆₀₀ of 1. This cell suspension was used to prepare 1:500 dilutions, corresponding to approximately 2×10⁶ microbial cells per ml.

To evaluate MICs, 96-well plates were prepared with the test antimicrobial peptides. Each well contained 100 μl microbial cell culture, 90 μl culture medium, and 10 μl peptide or control at the corresponding dilution. The plates were incubated overnight (ON) at the optimal growth temperature for each tested organism (Table 1). Results were visually evaluated and MICs determined based on growth or no-growth.

TABLE 1
Organisms that were tested in MIC assays for a range of antimicrobial
peptides. Given are the species name, classification as well as
the growth conditions. DSM refers to the ordering number at the
German Collection of Microorganisms and Cell Cultures GmbH (DSMZ,
Braunschweig, Germany). SF refers to organisms obtained from the
Jena Microbial Resource Collection (JMRC, Jena, Germany).

| | | | | Growth |
|---|---|---|---|---|
| | Ordering | | Growth | temper- |
| Species | number | Classification | medium | ature |
| Pantoea | JUB-CC-3253 | Gram-negative | TSY | 37° C. |
| agglomerans | | bacterium | | |
| Staphylococcus | DSM 11729 | Gram-positive | TSY | 37° C. |
| aureus MRSA | | bacterium | | |
| Listeria | DSM 102976 | Gram-positive | TSY | 37° C. |
| monocytogenes | | bacterium | | |
| Candida albicans | DSM 1386 | Yeast | YPD | 30° C. |
| Ceratosystis | DSMZ 63054 | Fungus | PDA | 18° C. |
| paradoxa | | | | |
| Botrytis cinerea | DSMZ 877 | Fungus | PDA | 24° C. |
| Leptosphaeria | SF 70 | Fungus | PDA | 18° C. |
| maculans | | | | |
| Mycosphaerella | DSMZ 62763 | Fungus | PDA | 30° C. |
| pinodes | | | | |
| Sclerotinia | SF 79 | Fungus | PDA | 18° C. |
| sclerotiorum | | | | |
| Verticillium | SF 76 | Fungus | PDA | 28° C. |
| dahlia | | | | |

MIC assays were conducted in at least two independent replicates and with different peptide starting concentrations. Pre-tests showed, that none of the conducted steps to purify the tested proteins impacted obtained MICs.

Results: All the peptides according to the invention showed strong antimicrobial activity against all tested organisms.

The MIC values obtained for the peptides according to SEQ ID N°1, SEQ ID N°2, SEQ ID N° 4, SEQ ID N° 6 and SEQ ID N° 7 against fungal species are shown in Table 2.

All the peptides of the invention were more efficient in inhibiting fungi growth at a lower concentration than the peptides of the prior art.

TABLE 2
Results of MIC assays with fungal species as tester organisms and the peptides of the invention (SEQ ID
No 1, No 2, No 4) and prior art peptides (SEQ ID No 6 and No 7). Clotrimazole was used as a reference.

| | Minimal inhibitory concentration (μg/ml) | | | | | |
|---|---|---|---|---|---|---|
| | | Peptide of | Peptide of | Peptide of | Prior art | Prior art |
| | | the invention | the invention | the invention | peptide | peptide |
| | | according to | according to | according to | according to | according to |
| Fungus | Clotrimazole | SEQ ID No 1 | SEQ ID No 2 | SEQ ID No 4 | SEQ ID No 6 | SEQ ID No 7 |
| Candida | 3.1 | 0.4 | 3.1 | 0.4 | 13.1 | 12.75 |
| albicans | | | | | | |
| Ceratosystis | 0.4 | 5 | 6.25 | 1 | 4.8 | 4.7 |
| paradoxa | | | | | | |
| Botrytis cinerea | 6.25 | 6.25 | 6.25 | 3.1 | 14.5 | 12.75 |
| Leptosphaeria | 0.4 | 3.75 | 6.25 | 4.1 | 37.9 | 9.4 |
| maculans | | | | | | |
| Mycosphaerella | 3.1 | 1.9 | 4.5 | 2 | 13.12 | 10.68 |
| pinodes | | | | | | |
| Sclerotinia | 25 | 10.25 | 19.3 | 1.5 | 27.2 | 23.5 |
| sclerotiorum | | | | | | |
| Verticillium | 1.56 | 1.6 | 3.1 | 1.55 | 14.5 | 12.7 |
| dahliae | | | | | | |

The MIC values obtained for the peptides according to SEQ ID N° 1, SEQ ID N° 2, SEQ ID N° 4, SEQ ID N° 6 and SEQ ID N° 7 against bacterial species are shown in Table 3. All the peptides of the invention inhibited bacterial growth, and the peptides according to SEQ N° 1, N° 2, and N° 4 were more efficient than the prior art peptides in all tested species.

TABLE 3
Results of MIC assays with bacterial species as tester organisms
and the peptides of the invention (SEQ ID No 1, No 2, and
No 4) and prior art peptides (SEQ ID No 6 and No 7). Gentamycin
and Streptomycin were used as references.

| | Minimal inhibitory concentration (μg/ml) | | | | | | |
|---|---|---|---|---|---|---|---|
| | | | SEQ | SEQ | SEQ | SEQ | SEQ |
| | Genta- | Strepto- | ID | ID | ID | ID | ID |
| Bacteria | mycin | mycin | No 1 | No 2 | No 4 | No 6 | No 7 |
| Staphylococcus | 0.8 | >125 | 3.1 | 7.1 | 1.8 | 13 | 13.6 |
| aureus | | | | | | | |
| Listeria | 0.4 | 7.8 | 3.1 | 7.1 | 1.8 | 3.25 | 13.6 |
| monocytogenes | | | | | | | |
| Pantoea | 1.56 | NA | 1.1 | 1.9 | 1 | 7.9 | 9.45 |
| agglomerans | | | | | | | |
| E. coli | 1 | NA | 3.1 | 6.2 | NA | NA | NA |

### Example 5: Peptides Stability in Bodily Fluids

### Sheep Blood Assay

In vitro experiments were conducted to test the behavior of the peptides as disclosed herein in blood, against C. albicans. C. albicans isolates from human blood were used (DSMZ 6569). The peptide according to the SEQ ID N° 1 was adjusted to a concentration of 1 mg/ml and mixed with sheep blood in equal parts. This mixture was incubated overnight at 30° C. To remove solid parts of the blood, the mixture was centrifuged and the obtained plasma supernatant was used for MICs.

### Urine Assay

In vitro experiments were conducted to test the behavior of the peptides as disclosed herein in urine, against C. albicans. The peptide according to the SEQ ID N° 1 was adjusted to a concentration of 1 mg/ml and mixed with synthetic urine or human urine in equal parts. These mixtures were incubated overnight at 30° C. To remove solid parts of the urine, the mixture was centrifuged and the obtained supernatant was used for MICs.

### Cow Milk Assay

In vitro experiments were conducted to test the behavior of the peptides as disclosed herein in 3.5% fat cow milk, against Listeria monocytogenes. The peptide according to the SEQ ID N° 1 was adjusted to a concentration of 1 mg/ml and mixed with cow milk in equal parts. This mixture was incubated overnight at 37° C. To remove solid parts of the milk, the mixture was centrifuged and the obtained supernatant was used for MICs.

Results: A decrease in the ability to inhibit C. albicans cell growth, was observed when the peptides were mixed with sheep blood. However, the peptides as described herein were still active at 25 μg/ml in sheep blood (Table 4).

The ability to inhibit the growth of C. albicans cell growth was to a lesser extent impacted by the mixing of the peptide with urine. In synthetic urine, the efficient antimicrobial activity was achieved at 2 μg/ml peptide and in human urine at 5 μg/ml peptide, compared to 0.4 μg/ml in control conditions (Table 4).

Similarly, when the peptides were mixed with milk, the concentration that inhibited the growth of L. monocytogenes increased to 80 μg/ml, from 3.2 μg/ml in milk-free conditions (Table 4).

The peptides did not completely lose their ability to inhibit bacteria and yeast growth in bodily fluids, suggesting that they are suitable for therapeutic use.

TABLE 4
Results of MIC assays when the peptide of the invention
(SEQ ID No 1) was mixed with sheep blood, urine, or cow
milk. The control is the peptide with no bodily fluid,
and caspofungin and tetracycline are used as references.

| | | Minimal inhibitory |
|---|---|---|
| Organism tested | Condition | concentration (μg/ml) |
| Candida albicans | Control | 0.4 |
| | Caspofungin | 0.5 |
| | Sheep blood | 25 |
| | Synthetic urine | 2 |
| | Human urine | 5 |
| Listeria monocytogenes | Control | 3.1 |
| | Tetracycline | 1.6 |
| | Cow milk | 80 |

### Example 6: MIC Assay in Soil Slurry

In vitro experiments were conducted to test the behavior of the peptides as disclosed herein in soil slurry, against Erwinia amylovora. Soil slurry was generated by mixing 5 g soil in 20 ml water. This mixture was incubated for 1 h at an overhead shaker. The solid parts were removed by centrifugation and the obtained supernatant, referred to as soil slurry, was used for the MIC assay. Equal volumes of the peptide according to SEQ ID N°1 (8 mg/ml) and soil slurry were mixed and incubated overnight at 28° C. The mixture was used for MIC against Erwinia amylovora.

Results: The peptides as described herein inhibited the bacteria growth at 25 μg/ml in soil slurry compared to 15 μg/ml in control conditions. As they do not completely lose their ability to inhibit bacterial growth in soil, the peptides as described herein are suitable for agricultural use.

### Example 7: Peptides Applicability on Technical Surfaces

Experiments were conducted to test the behavior of the peptides as disclosed herein on technical surfaces, against Candida albicans.

Bio-film formation of this organism was tested for ceramic (1100 μm pore size), stainless steel, and silicon surfaces in a biofilm reactor. Four stalks of discs, each containing discs of the 3 surfaces, were entered into each reactor and screwed tight. The systems were autoclaved with all discs mounted. 297 ml of SU medium were filled into each of the reactors, and 3 mL of a C. albicans (vaginal isolate) preculture in SU (OD₆₀₀=0.1) were added. The biofilm reactors were incubated at 30° C. and 200 rpm stirring. Three bioreactor systems were run in total: a control, a treatment with peptides enriched cocoa extract from day 0, and a treatment with said extract from day 3. The cocoa extract had a final total protein concentration of 100 μg/mL.

On days 1, 3, 5, and 7, a stalk of discs was removed from each reactor. The discs were unscrewed and submerged in 2 mL of SU. These liquids were diluted in SU medium and a dilution series of 10⁻¹-10⁻⁵ was prepared. The dilutions were plated on a YPD solid medium and were incubated at 30° C. for 48 h. The colonies formed on the plates were counted and the colony forming units (CFU)/cm² disc was calculated.

Results: No growth of C. albicans was observed on all tested surfaces. These results suggest that the peptides as disclosed herein are highly stable and effective for use as surface disinfectants or decontaminants.

### Experiment 8: In Vitro Assay in Yeast and Mammalian Cells

In order to investigate whether the growth-inhibiting effect of the peptides is of microstatic or microbicidal nature, a lactate dehydrogenase (LDH) release assay was performed on C. albicans (vaginal isolate) as a model organism of choice and on human keratinocytes (HaCaT) cells. LDH is an enzyme present in many cell types. Damage to the cell membrane results in the release of LDH into the cell culture medium, which can subsequently be detected and quantified via a coupled enzymatic color reaction.

HaCaT cells were grown in DMEM medium supplemented with 4.5 g/L glucose, 2 mM L-glutamine and 10% fetal bovine serum (FCS) at 37° C. and 8% CO₂. The cells were grown in 75 cm² cell culture flasks with approximately 20 ml of DMEM. The media were changed twice a week. The cells were passaged at a ratio of 1:10 once confluency of more than 90% was reached. The passaging solution consisted of a 1:1 mixture of EDTA (0.05% stock) and trypsin (0.1% stock) in PBS without Ca²⁺ and Mg²⁺.

The LDH cytotoxicity assay CyQUANT™ (Invitrogen, no. C20300 and C20301) was conducted according to the protocol provided by the company. The optimal seeding density was determined to be 10,000 cells per 100 μL for HaCaT or C. albicans cells according to a standard protocol as described in the kit. The cells were exposed to peptide according to SEQ ID N° 2 for 1 h or overnight with concentrations ranging from 1.95 μg/ml to 250 μg/ml and the LDH release was detected. The following formula provided by the company was used to calculate cytotoxicity: % Cytotoxicity=[(Peptide−treated LDH activity)−(Spontaneous LDH activity)(Maximum LDH activity)−(Spontaneous LDH activity)]*100, with Spontaneous LDH activity being measured in the untreated (water-added) sample and Maximum LDH activity being measured in the with the lysing agent provided by the kit supplier.

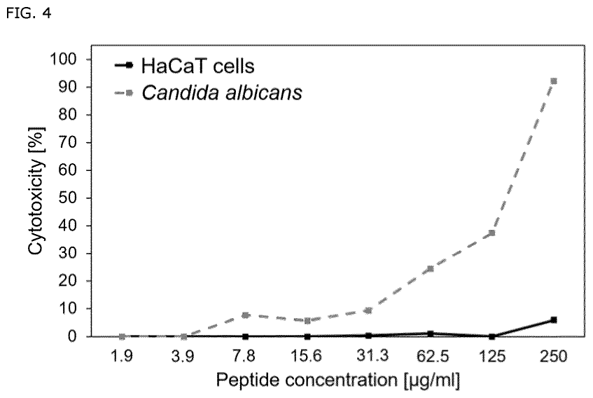

Results: The detected cell death of the target organism C. albicans was linear from 0 to 250 μg/ml up to a cytotoxicity of 92% at the highest tested protein concentration (FIG. 4). In contrast, little to no release of LDH was induced upon treatment of HaCaT cells with the peptide. The cellular cytotoxicity of the peptide to HaCaT cells was between 0% and 1% up to a peptide concentration of 125 μg/ml and reached 6% at a peptide concentration of 250 μg/ml (FIG. 4).

It is supposed that the present invention is not restricted to any form of realization described previously and that some modifications can be added to the presented example of fabrication without reappraisal of the appended claims. For example, preferred embodiments of the present invention are directed to CCN-51 variety of Theobroma cacao, but any cocoa variety which yields vicilin in a sufficient amount for further processing can be used without departing from the scope of the present invention.

### Example 9: Peptides Extraction from Non-Fermented Cocoa Beans

The peptides as disclosed herein were isolated from non-fermented but well-dried cocoa beans. The cocoa beans were crushed mechanically with a bean breaker in order to obtain a mixture of nibs and shells. The shells were removed and the deshelled nibs were transferred to an expeller. Thereby, a mixture of butter and slurry was obtained, which was separated by centrifuging at 16,600×g for 10 minutes at 40° C. The butter fraction collected at the top was discarded, while the solid slurry pellet was pulverized and stored at 4° C. until further use.

The peptides were then extracted in 3 manners.

### Method 1: PEB (Protein Extraction Buffer) Extraction

The pulverized slurry pellet was pre-heated in a water bath at 50° C. for 10 min and then dissolved in protein extraction buffer (100 mM Tris, 1% SDS and 1% DTT, pH 8.1), which was added in a 1:4 w/v pellet to buffer ratio. The dissolved slurry pellet was then incubated for 1 h at room temperature under agitation, followed by centrifugation at 16 600×g for 20 min. The peptide-containing liquid phase was collected and purified via dialysis or ultrafiltration (Centricon Plus-70, Merck Chemicals GmbH, an affiliate of Merck KGaA, Darmstadt, Germany) to remove the components of the PEB.

### Method 2: PEB Extraction Followed by Acid Precipitation

The pulverized slurry pellet was treated as described in method 1, namely, the pulverized slurry pellet was pre-heated in a water bath at 50° C. for 10 min and then dissolved in PEB. The dissolved slurry pellet was then incubated for 1 h at room temperature under agitation, followed by centrifugation at 16,600×g for 20 min. The peptide-containing liquid phase was collected and subjected to acid precipitation to enrich the peptide of the invention in the protein mix based on its isoelectric point. Thereby, the pH of the protein extracts was lowered to 6.0 with 1 M citric acid, resulting in a color change from dark grey to red. This mixture was then subjected to centrifugation at 16,600×g for 10 min. The supernatant was discarded and the protein pellet was resuspended in the PEB using sonication to properly dissolve the pellet. After this, harmful substances were removed via ultrafiltration with a 5 kDa MWCO (Centricon Plus-70, Merck Chemicals GmbH, an affiliate of Merck KGaA, Darmstadt, Germany).

### Method 3: Heat Extraction

The pulverized slurry pellet was heated to 60° C. in tap water in a 1:4 w/v pellet to water ratio for 10 min under stirring. The obtained mixture was centrifuged at 16,600×g for 20 min at 10° C.

The peptide-containing supernatant, obtained after extraction with any of the methods 1 to 3, was collected and quantified or analyzed by SDS-PAGE and MICs. The protein extract was lyophilized or vacuum centrifuged for up-concentration prior to its application.

1. A peptide consisting of a first peptide and optionally a signal peptide, wherein said first peptide has an amino acid sequence according to SEQ ID NO. 1 or SEQ ID NO. 2 or that exhibits at least 98% sequence identity to SEQ ID NO. 2 or that differs with maximally 2 amino acids from the amino acid sequence according to SEQ ID NO. 2.

2. (canceled)

3. The peptide according to claim 1, wherein said first peptide is fused to said signal peptide at its N-terminus; and/or
wherein said peptide comprises said signal peptide, said signal peptide having an amino acid sequence that exhibits at least 95% sequence identity to SEQ ID NO. 3 or having an amino acid sequence according to SEQ ID NO. 3.

4. (canceled)

5. The peptide according to claim 1, wherein said peptide has antibacterial and/or antifungal activity; or
wherein said peptide has antibacterial and/or antifungal activity against Gram-positive and Gram-negative bacteria and/or fungi; or
wherein said peptide has antibacterial and/or antifungal activity against hyphal fungi and yeasts.

6. (canceled)

7. The peptide according to claim 1, wherein said peptide is derivable from or isolated from Theobroma cacao; or,
wherein said peptide is derivable from or isolated from Theobroma cacao variety CCN-51.

8. The peptide according to claim 1, wherein said peptide is a recombinant or a synthetic peptide.

9. A composition comprising:
a peptide consisting of a first peptide and optionally a signal peptide, wherein said first peptide has an amino acid sequence exhibiting at least 95% sequence identity to SEQ ID NO. 1 or SEQ ID NO. 2 or that differs with maximally 5 amino acids from the amino acid sequence according to SEQ ID NO. 1 or SEQ ID NO. 2; and
an excipient.

10. The composition according to claim 9, wherein said first peptide is fused to said signal peptide at its N-terminus; and/or
wherein said peptide comprises said signal peptide, said signal peptide having an amino acid sequence exhibiting a sequence identity of at least 95% to SEQ ID NO. 3 or having an amino acid sequence according to SEQ ID NO. 3.

11. The composition according to claim 9, wherein said composition is a liquid, a semisolid, a solid, or in gaseous form and/or wherein said composition is in a dosage form of a tablet, capsule, powder, granulate, aerosol, paste, syrup, suspension, emulsion or solution.

12. (canceled)

13. (canceled)

14. A method comprising:
contacting an effective amount of the composition according to claim 9 with a crop, a feed or food, a cosmetic, a pharmaceutical, a preservative, or a surface to control, reduce, or eliminate a pathogen therein; or
administering a therapeutically effective amount of the composition according to claim 9 to a subject in need thereof to prevent or treat a pathogenic infection in the subject.

15. The method according to claim 14, wherein said first peptide is fused to the signal peptide at its N-terminus; and/or,
wherein said peptide comprises the signal peptide, said signal peptide having an amino acid sequence exhibiting a sequence identity of 95% to SEQ ID N° 3 or having an amino acid sequence according to SEQ ID N° 3.

16. (canceled)

17. The method according to claim 14, wherein said peptide has antibacterial and/or antifungal activity; or
wherein said peptide has antibacterial and/or antifungal activity against Gram-positive and Gram-negative bacteria and/or fungi; or
wherein said peptide has antibacterial and/or antifungal activity against hyphal fungi and yeasts.

18. (canceled)

19. The method according to claim 14, wherein the method controls a pathogen in a plant or in a crop.

20. The method according to claim 14, comprising administering a therapeutically effective amount of the composition to a subject in need thereof to prevent or treat a bacterial and/or a fungal infection.

21. A method for producing a peptide consisting of a first peptide and optionally a signal peptide, said first peptide having an amino acid sequence exhibiting at least 95% sequence identity to SEQ ID NO. 1 or SEQ ID NO. 2 or that differs with maximally 5 amino acids from the amino acid sequence according to SEQ ID NO. 1 or SEQ ID NO. 2, and wherein said peptide is produced by peptide recombination techniques, by synthesis, or by extraction or precipitation from Theobroma cacao.

22. The method according to claim 21, wherein the peptide is extracted from cocoa beans slurry.

23. A vector comprising a coding sequence for the peptide according to claim 1, wherein said vector is designed to allow expression of said peptide in an expression system.

24. A transgenic plant expressing a peptide according to claim 1.

25. A method of producing the transgenic plant of claim 24, wherein said method comprises the step of introducing a nucleic acid encoding for said peptide into a plant cell thereby producing a transformed plant cell expressing said peptide and regenerating the transformed plant cell into a transgenic plants.

26. A seed of a plant coated with the peptide or composition according to claim 1.

27. The vector according to claim 23, wherein:
the first peptide is fused to the signal peptide at its N terminus; and/or
the peptide comprises the signal peptide, said signal peptide having an amino acid sequence exhibiting at least 95% sequence identity to SEQ ID NO. 3 or having an amino acid sequence according to SEQ ID NO. 3.
